(19) United States
(12) Patent Application Publication
(10) Pub. No.: US 2023/0270799 A1
(43) Pub. Date: Aug. 31, 2023

(54) BIOMATERIAL COMPRISING BACTERIAL CELLULOSE AND PROBIOTICS AND USES THEREOF

(71) Applicant: UNIVERSIDAD DE GRANADA, Granada (ES)

(72) Inventors: José Manuel DOMÍNGUEZ VERA, Armilla, Granada (ES); José Manuel DELGADO LÓPEZ, Armilla, Granada (ES); Ana GONZÁLEZ GARNICA, Granada (ES); Gloria Belén RAMIREZ RODRIGUEZ, Granada (ES); Natividad GÁLVEZ RODRÍGUEZ, Cenes de la Vega, Granada (ES); Laura SABIO RODRÍGUEZ, Granada (ES)

(21) Appl. No.: 18/004,082

(22) PCT Filed: Jul. 1, 2021
(86) PCT No.: PCT/EP2021/068166

§ 371 Date: Jan. 3, 2023

(30) Foreign Application Priority Data
Jul. 1, 2020 (ES) P202030674

(51) Int. Cl. A61K 35/747; A61P 31/04; A61K 35/745; A01N 63/20; A01N 43/16; A61K 31/717; C12N 1/20; A23P 20/10; A23L 33/135; C12N 11/04

(52) U.S. Cl. CPC A61K 35/747; A61P 31/04; A61K 35/745; A01N 63/20; A01N 43/16; A61K 31/717; C12N 1/20; A23P 20/105; A23L 33/135; C12N 11/04; A23Y 2220/35; A23Y 2220/37

(57) ABSTRACT

The invention relates to biomaterials, which comprise a bacterial cellulose matrix and probiotics entrapped in said bacterial cellulose. The invention also relates to methods for obtaining the biomaterial, as well as uses of the biomaterial in medicine. It also relates to coated food products wherein the coat is composed of said biomaterial wherein the coat acts to prevent the proliferation of undesired bacteria. It also relates to packaged medical devices, wherein the package comprises said biomaterial also preventing growth of pathogenic bacteria.

### FIELD OF THE INVENTION

The invention relates to the field of probiotics provided within a biomaterial, and their use in therapy, in particular for the treatment or prevention of bacterial infections. It also relates to their use as coat or package of food products and medical devices to impede the development of pathogen bacteria.

### BACKGROUND OF THE INVENTION

According to the World Health Organization, the dramatic increase of antibiotic resistant bacteria is one of the biggest threats to global health. Antibiotic resistance causes around 700.000 deaths per year worldwide and this could lead to 10 million deaths by 2050. Given the extremely urgent situation, new antibiotic-free approaches to address bacterial infections are needed.

A hopeful alternative could be the use of probiotics. Probiotics are live microorganisms intended to provide health benefits by restoring the microbiome or through excreted anti-pathogenic compounds as bacteriocins or hydrogen peroxide. Nonetheless, the viability of naked probiotic and thus its beneficial health effects are endangered during the process of nesting and proliferating in the hostile environment of the targeted tissue. Therefore, one of the keys for health application of probiotics is the choice of the appropriate material serving as a matrix to house live probiotics.

Bacterial cellulose (BC) has been widely explored for biomedical applications, in particular as a wound dressing material. Indeed, BC provides optimum moisture balance to dry wounds, absorbs wound exudates, serves as an effective physical barrier against any external infection and does not adhere to the surface of the wound and has no damaging effect on tissue upon removal. In vivo studies of wound healing have demonstrated that BC-based materials show faster epithelialization and regeneration than other commercially available products. However, BC itself has no activity against bacterial infection and attempts to incorporate drugs in cellulose to treat these infections did not work properly. Regarding other biomedical applications of BC, different to wound cures, its major obstacle relies on the limited surface charge and lack of functional groups for anchoring of bioactive compounds. It is insoluble in water and in the most common organic solvents, so its efficient functionalization with active chemical groups for the entrapment or grafting bioactive compounds, such as drugs or proteins is very difficult. To generate BC derivatives with better properties for biomedical applications, two approaches have been envisaged: BC functionalization and hybridization of BC with other polymers. However, no definitive results have been reported so far. Thus, the use of BC as carrier for therapeutically active ingredients has not provided successful results so far.

Thus, new methods to efficiently treat or prevent infections, alternative to antibiotics, are required in the field.

### SUMMARY OF THE INVENTION

The inventors have developed a biomaterial which comprises bacterial cellulose essentially free of cellulose-producing bacteria, and probiotics. Said biomaterial is obtained by culture of aerobic cellulose-producing bacteria (in particular Acetobacter xylinum bacteria) together with facultative anaerobic probiotics (in particular Lactobacillus fermentum, Lactobacillus gasseri or Bifidobacterium breve bacteria), under aerobic conditions first, and then under anaerobic conditions.

The inventors have shown that the entrapped probiotics in the bacterial cellulose are alive and metabolically active. Moreover, they have observed that the biomaterials severely affect proliferation of pathogenic bacteria commonly involved in the development of skin and wound infections. In particular, they inhibit proliferation of Staphylococcus aureus (SA) and Pseudomonas aeruginosa (PA) in tryptic soy agar (TSA) or Tryptic soy broth (TSB) culture medium, which are particularly favorable for pathogenic proliferation but not for probiotic proliferation.

Thus, in a first aspect, the invention relates to a biomaterial comprising a bacterial cellulose matrix and probiotics entrapped in said matrix.

In a second aspect, the invention relates to a method for obtaining the biomaterial of the first aspect, comprising:
(i) culturing aerobic bacteria that produce cellulose simultaneously with facultative anaerobic probiotics or aerotolerant anaerobic probiotics under conditions suitable for the production of cellulose by the bacteria that produce cellulose, thereby resulting in a cellulose matrix containing the bacteria and the probiotics and,
(ii) incubating the cellulose matrix obtained in step (i) in a culture medium that provides conditions which are suitable for the proliferation of the probiotics in said matrix and which are not suitable the proliferation of the aerobic bacteria.

A third aspect of the invention relates to a biomaterial obtained by the method of the second aspect of the invention.

A fourth aspect of the invention relates to the biomaterial of the first or third aspect of the invention, for use in medicine.

A fifth aspect of the invention relates to the biomaterial of the first or third aspect of the invention, for use in the treatment of a wound or of a bacterial infection.

A sixth aspect relates to a pharmaceutical composition comprising the biomaterial of the first or third aspect of the invention, and a pharmaceutically acceptable carrier.

A seventh aspect of the invention relates to a coated food product which comprises:
(i) a biomaterial according to the first or third aspect of the invention, and
(ii) an edible filling composition,

wherein the biomaterial (i) coats the filling composition (ii).

An eighth aspect relates to a packaged medical device wherein the device is packaged in a container which comprises a biomaterial of the first or third aspect of the invention.

A ninth aspect relates to the use of the biomaterial of the first or third aspect of the invention as a coat in a coated food product.

A tenth aspect of the invention relates to the use of a biomaterial of the first or third aspect of the invention for the packaging of a medical device.

### DETAILED DESCRIPTION OF THE FIGURES

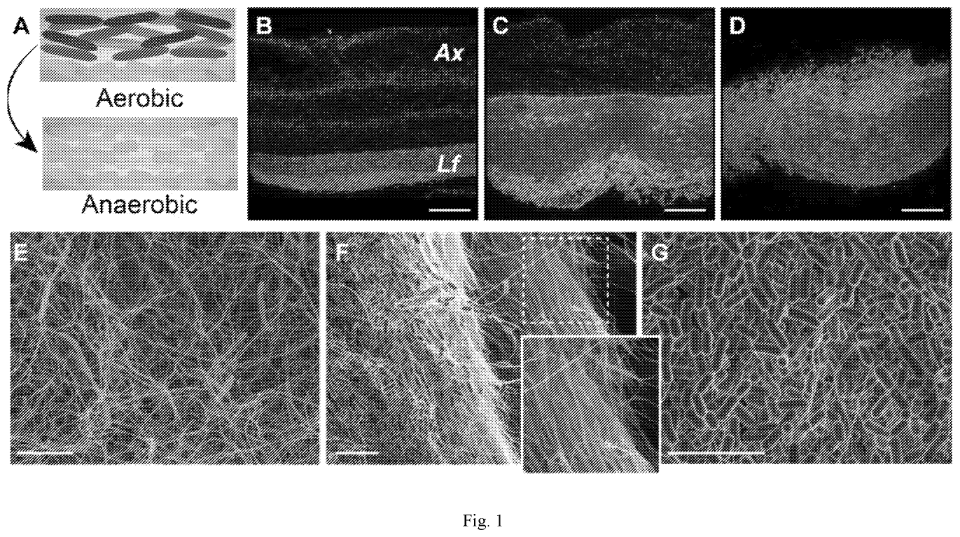

FIG. 1. Characterization of probiotic cellulose. (A) Graphical description of BC obtained under aerobic conditions (top) and probiotic cellulose produced by switching to anaerobic conditions (bottom). (B-D) Dark-field optical micrographs of cross-sections of Gram-stained cellulose films showing the gradual invasion of the probiotics as a function of increasing incubation time (from left to right). Scale bar=100 μm. (E) SEM micrograph of the air-exposed surface of cellulose co-cultured with Ax and Lf in aerobic conditions (BC). Note that most of the bacteria present the typical fibrous morphology of Ax. (F) SEM micrograph of the cross-section of the two-sided material formed under anaerobic conditions (24 hours of incubation): one side contains Ax (right) and the other Lf (left). (G) SEM micrograph of cellulose co-cultured with Ax and Lf under anaerobic conditions (48 hours of incubation). In this case, both surfaces (exposed to either air or solution) provided similar results. Note that all the bacteria exhibit the typical morphology of Lf Scale bars=5 μm.

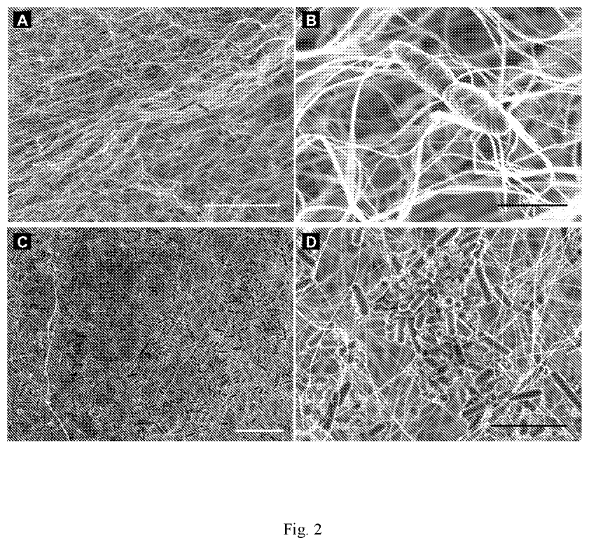

FIG. 2. Scanning electron microscopy of Lg-cellulose. (A,B) Lg-cellulose after incubation in aerobic conditions. (C,D) Lg-cellulose after 48 h of incubation in anaerobic conditions. Scale bars: A, C, D=5 μm, B=1 μm.

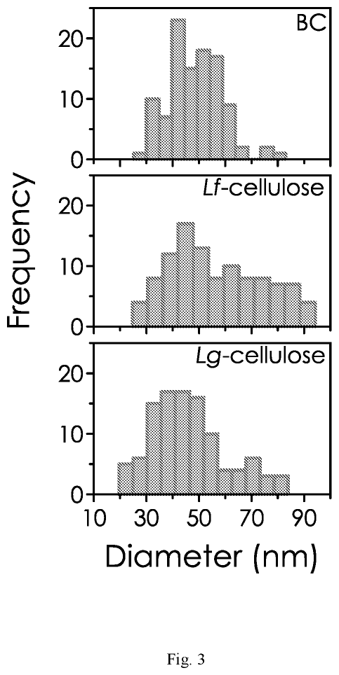

FIG. 3. Size distribution of cellulosic materials. Histogram of fibril diameters of bacterial cellulose (BC), Lf-cellulose and Lg-cellulose. The diameter of 100 fibers from different SEM images were measured at each condition.

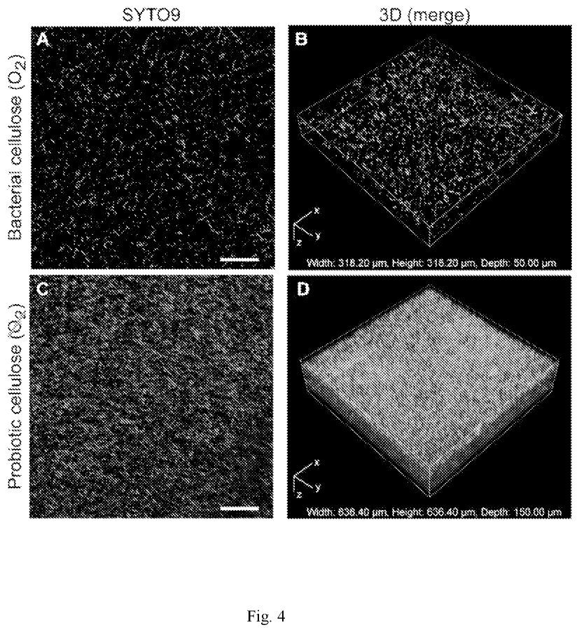

FIG. 4. Live/dead viability assays. CLSM images of BC co-cultured with Ax and Lf under aerobic, (A,B), and then, anaerobic conditions, (C-D). Panels A,C show the bacteria stained with SYTO 9 (live bacteria). The 3D maps (B,D) are representative of the merged images with bacteria stained with SYTO 9 (live bacteria) and propidim iodide (dead bacteria, negligible). Scale bars=50 μm.

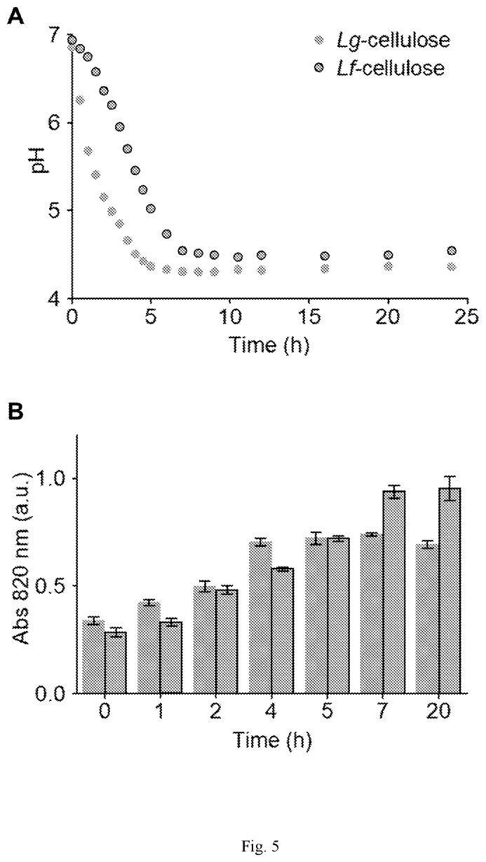

FIG. 5. Metabolic activity of probiotic cellulose. (A) Time evolution of the pH of MRS media containing a film of probiotic cellulose. (B) Time dependence of the UV-vis absorbance at 820 nm of probiotic cellulose in contact with a solution containing POM. Data are expressed as mean with the corresponding standard deviation as error bars.

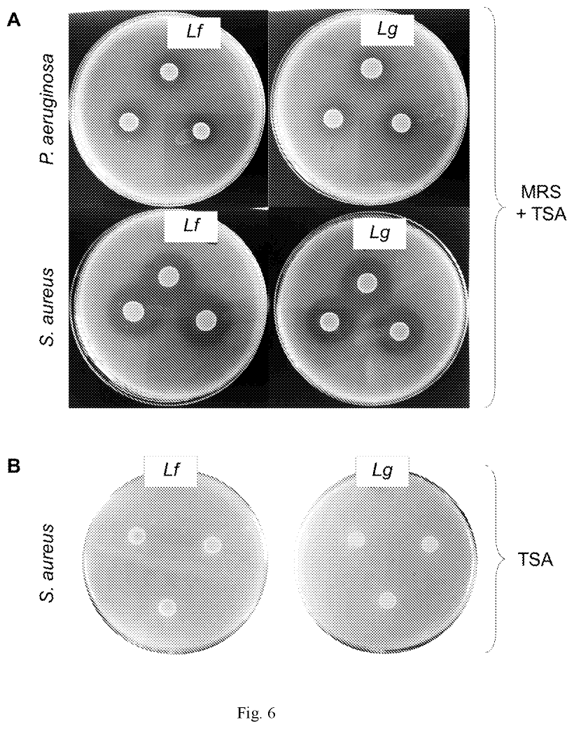

FIG. 6. Media-dependent inhibition activity of non-encapsulated Lf and Lg probiotics. (A) Inhibitory activity of the probiotics in MRS+TSA medium against SA and PA, and (B) in TSA against SA.

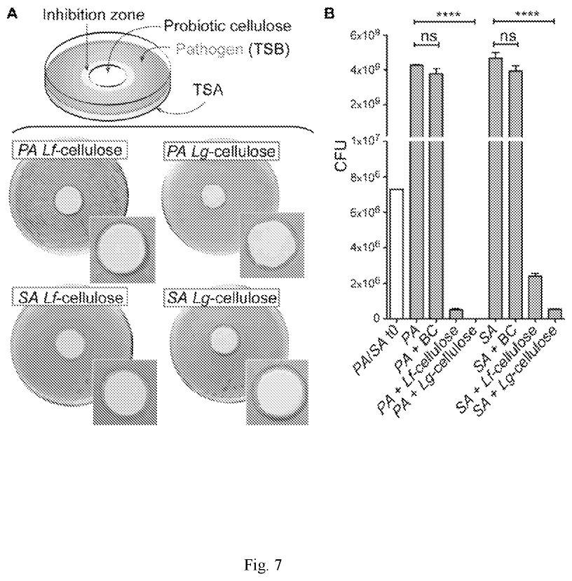

FIG. 7. Inhibitory and antibacterial activity of probiotic cellulose. (A) Diagram of the experimental protocol used to assess the inhibitory activity of probiotic cellulose (Lf- and Lg-cellulose) against Staphylococcus aureus (SA) and Pseudomonas aeruginosa (PA). Even though each pathogen was cultivated in an optimal medium, we observed clear inhibition zones around the probiotic cellulose for both PA and SA (see expanded views). (B) PA and SA survival after co-incubation with BC or probiotic celluloses (Lf- and Lg-cellulose) in tryptic soy broth (TSB). Asterisks and ns denote statistical significance (p<0.001) and no significance, respectively.

### DETAILED DESCRIPTION OF THE INVENTION

1—Biomaterial of the Invention

In a first aspect the invention relates to a biomaterial comprising a bacterial cellulose matrix and probiotics entrapped in said matrix.

Said biomaterial is herein referred to as the biomaterial of the invention.

The term “biomaterial”, as used herein, refers to a designed material or product, that is suitable to interact with biological tissues or with an organism, preferably with the human body, a particular organ of the human body, or a particular region of an organ of the human body.

The term “cellulose”, as used herein, refers to the term commonly known by an expert in the field. In particular, it refers to the homopolymer with the formula (C₆H₁₀O₅)ₙ. It consists in a linear chain of several hundred to many thousands of β(1→4) linked D-glucose units. It is part of the cell wall of green plants, algae, oomycetes, and can also be produced by some bacteria. Different crystalline structures of cellulose are known, corresponding to the location of hydrogen bonds between and within strands. Natural cellulose is cellulose I, with structures Iα (triclinic) and Iβ (monoclinic).

Cellulose Iα and cellulose Iβ have the same fiber repeat length (that is, unit cell dimension c, being 1.043 nm for the repeat dimer in crystallites inside the fiber and 1.029 nm in crystallites at the surface (Davidson T. C. et al., 2004, Carbohydrate research, 339: 2889-2893)) but differing displacements of the sheets relative to one another. The neighboring sheets of cellulose Iα (consisting of identical chains with two alternating glucose conformers -A-B-) are regularly displaced from each other in the same direction whereas sheets of cellulose Iβ (consisting of two conformationally distinct alternating sheets—the 2-OH and 6-OH groups both change orientations so altering the hydrogen bonding pattern—each made up of crystallographically identical glucose conformers) are staggered (Nishiyama. Y. et al., 2002, Journal of the American Chemical Society, 124: 9074-9082). The two crystal alomorphs can occur not only within the same sample of cellulose but also along a given microfibril. Cellulose Iα is considered to predominate in algal and bacterial cellulose, whereas cellulose Iβ is the dominant form in higher plants.

The expression “bacterial cellulose”, or “BC”, as used herein, refers to cellulose as defined above, produced by bacteria, preferably by bacteria from the genus Acetobacter, Gluconacetobacter, Komagataeibacter, Rhizobium, Agrobacterium, Enterobacter, Achromobacter, Azotobacter, Salmonella, Escherichia, and Sarcina. Whereas plant-derived cellulose chains are closely associated with hemicelluloses, lignin, and pectin, BC is free of other polymers. In addition, as indicated above, BC is primarily formed by cellulose Ia. The characteristics of the BC have been described in several documents known by an expert in the field, such as Moon R. J. et al. 2011, Chem. Soc. Rev., 40:3941-3994, Sulaeva I. et al., 2015, Biotechnology advances, 33:1547-1571 or Zhang. W. et al., 2018, Food Sci. Biotech. 27:705-713. Briefly, during its biosynthesis by bacteria, cellulose chains are polymerized by cellulose synthases A (CesA) from activated glucose. The single chains are then extruded through the bacterial cell wall by rosette terminal complexes into the external medium. The macromolecules assemble into hierarchically organized units as a complex, primarily forming subfibrils of 10-15 glucan chains that assemble to form microfibrils, assembled into microfibril bundles. The loosely assembled bundles then form cellulose ribbons comprised of about 1000 polyglucan chains. Continuous spinning of cellulose ribbons by bacteria leads to the formation of a highly pure 3-D structure of nanofibers stabilized by inter- and intra-fibrillar hydrogen bonds. This structural singularity of the BC fibrillated network results in unique mechanical characteristics. Said characteristics include a high degree of crystallinity (60-80%) and a high Young's modulus of 15-30 GPa and a high degree of polymerization (up to 8000). The fibers also show a high aspect ratio, considered to be generally greater than 50 (Moon R. J. et al. 2011, Chem. Soc. Rev., 40:3941-3994). This high aspect ratio of the fibers results in a high surface area which provides a great liquid loading capacity of up to 99 wt. %. In the case of water, about 90% of the water molecules are tightly bound to the large number of hydroxyl groups within the cellulose molecules. BC fibers have a greater specific area in comparison to plant derived cellulose fibers. Water absorbency of BC was more than 30% greater than for cotton gauze, and the drying time was 33% longer (Sulaeva I. et al., 2015, Biotechnology advances, 33:1547-1571). Morphology of the fibers can vary with the specific bacteria producing them and with the culturing conditions. Typically, Acetobacter microfibrils have a rectangular cross-section (6-10 nm by 30-50 nm), having primarily Iα crystal structure (Moon R. J. et al. 2011, Chem. Soc. Rev., 40:3941-3994). Methods for identifying BC and for determining the properties of BC are described in Zhang. W. et al., 2018, Food Sci. Biotech. 27: 705-713.

The expression “bacteria that produce cellulose”, as used herein, refers to any bacteria capable of producing the bacterial cellulose as defined above. They can be aerobic, anaerobic, or facultative anaerobic bacteria. Non-limiting examples of said bacteria include bacteria from the genus Acetobacter, Gluconacetobacter, Komagataeibacter, Rhizobium, Agrobacterium, Enterobacter, Achromobacter, Azotobacter, Salmonella, Escherichia, Pseudomonas, Alcaligenis or Sarcina. Methods allowing identifying bacteria that produce cellulose are well-known by an expert in the field. Non-limiting examples of said methods include culturing a specific bacterial strain or species of interest under conditions suitable for bacterial cellulose production, and in the absence of cellulose in the initial culture media. After a certain time, generally 3-10 days, the presence of bacterial cellulose in the culture medium is analyzed. In case bacterial cellulose is found, it is considered that the tested bacteria are indeed bacteria that produce cellulose. The BC Culture conditions allowing bacteria to produce cellulose are described in Materials and Methods below and the analyses of the bacterial cellulose present in the culture medium of bacterial cellulose that produce BC in Example 1 below. Methods allowing differentiating bacterial strains that produce BC from those that do not are also described in Masoaka S. et al., 1993, Journal of fermentation and bioengineering, 175: 18-22 or in Zhang. W. et al., 2018, Food Sci. Biotech. 27: 705-713.

The expression “bacterial cellulose matrix” as used herein refers to any sample of bacterial cellulose, which as defined above, is a 3-D structure of nanofibers stabilized by inter- and intra-fibrillar hydrogen bonds, which results in a fibrillated network, or matrix of fibers. As understood by a skilled person, said matrix comprises the cellulose fibers, bounds between cellulose fibers, and/or within the same cellulose fiber, and empty spaces, or pours.

The term “probiotics” as used herein, refers to microorganisms which when provided to a subject, preferably a human, they confer a health benefit to said subject.

Non-limiting examples of probiotics include bacteria from the genera Lactobacillus, Bifidobacterium, Lactococcus, Streptococcus, Enterococcus, Pediococcus, Leuconostoc, Bacillus, Escherichia.

The expression “probiotics entrapped in said matrix”, as used herein, refers to probiotics, or a population of probiotics, contained within the bacterial cellulose matrix, in particular within one or several pours of the matrix. Said probiotics cannot freely move in all directions within the matrix, although no chemical bound may exist between the probiotics and the matrix (i.e. between any region of a probiotic bacteria and a fiber of the matrix). Thus, in order for the probiotics to get out of the matrix, or to reach the external surface of the matrix, an external force needs to be applied to the matrix, or a liquid needs to be applied to the matrix with a certain pressure, so that the probiotics move within the matrix until they reach the external surface of the matrix. However, as understood by a skilled person, the entrapped probiotics can proliferate within the matrix.

In a particular embodiment, the probiotics are not chemically bound to the matrix, i.e. no chemical bound exists between any region of the probiotics and any fiber of the matrix.

In a particular embodiment, the probiotics entrapped in the matrix of bacterial cellulose is a population of bacteria from a single species of bacteria.

In another particular embodiment, probiotics entrapped in the matrix of bacterial cellulose is a population of bacteria from a single bacterial strain.

In some embodiments, the probiotics entrapped in the matrix of bacterial cellulose is a population of bacteria from several species of bacteria. For example, it is a population of bacteria form 1, 2, 3, 4, 5, 6, 7, 8, 9, 10, 11, 5, 17, 20 different species.

In some embodiments, the probiotics entrapped in the matrix of bacterial cellulose is a population of bacteria from several bacterial strains. For example, it is a population of bacteria form 1, 2, 3, 4, 5, 6, 7, 8, 9, 10, 11, 5, 17, 20 different bacterial strains.

In a particular embodiment, the amount of probiotics comprised in the biomaterial of the invention is of about 1×10⁷, 5×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 5.5×10¹⁰, 6×10¹⁰, 6.2×10¹⁰, 6.5×10¹⁰, 6.7×10¹⁰, 7×10¹⁰, 7.2×10¹⁰, 7.5×10¹⁰, 7.7×10¹⁰, 8×10¹⁰, 8.1×10¹⁰, 8.2×10¹⁰, 8.3×10¹⁰, 8.4×10¹⁰, 8.5×10¹⁰, 8.6×10¹⁰, 8.7×10¹⁰, 8.8×10¹⁰, 8.9×10¹⁰, 9×10¹⁰, 9.1×10¹⁰, 9.2×10¹⁰, 9.3×10¹⁰, 9.4×10¹⁰, 9.5×10¹⁰, 9.6×10¹⁰, 9.7×10¹⁰, 9.8×10¹⁰, 9.9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.2×10¹¹, 2.5×10¹¹, 2.7×10¹¹, 3×10¹¹, 3.5×10¹¹, 4×10¹¹, 4.5×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 5×10¹³, 1×10¹⁴ 5×10¹⁴, 1×10¹⁵ CFU of probiotic bacteria per mg of BC, preferably about 8.7×10¹⁰ CFU of probiotic bacteria per mg BC, more preferably about 9.2×10¹⁰ CFU of probiotic bacteria per mg of BC, yet more preferably about 1×10¹¹ CFU of probiotic bacteria per mg of BC, even yet more preferably about 1.2×10¹¹ CFU of probiotic bacteria per mg of BC, even more preferably about 1.4×10¹¹ CFU of probiotic bacteria per mg of BC, even yet more preferably about 1.7×10¹¹ CFU of probiotic bacteria per mg of BC. In a preferred embodiment, the amount of probiotics comprised in the biomaterial of the invention is of 1.2×10¹¹ CFU of probiotic bacteria per mg of BC. In another preferred embodiment, the amount of probiotics comprised in the biomaterial of the invention is of 1×10¹¹ CFU of probiotic bacteria per mg of BC.

In a particular embodiment, the amount of probiotic bacteria comprised in the biomaterial of the invention is of at least any of the CFU of probiotic bacteria per mg of BC is as indicated above. In a preferred embodiment, the amount of probiotic bacteria comprised in the biomaterial of the invention is of at least 8.7×10¹⁰ CFU of probiotic bacteria per mg BC, preferably at least 9.2×10¹⁰ CFU of probiotic bacteria per mg of BC, more preferably at least 1×10¹¹ CFU of probiotic bacteria per mg of BC, even more preferably at least 1.2×10¹¹ CFU of probiotic bacteria per mg of BC, yet more preferably at least 1.4×10¹¹ CFU of probiotic bacteria per mg of BC, even yet more preferably at least 1.7×10¹¹ CFU of probiotic bacteria per mg of BC. In a preferred embodiment, the amount of probiotics comprised in the biomaterial of the invention is of at least 1.2×10¹¹ CFU of probiotic bacteria per mg of BC. In another preferred embodiment, the amount of probiotics comprised in the biomaterial of the invention is of at least 1×10¹¹ CFU of probiotic bacteria per mg of BC.

In another particular embodiment, the amount of probiotics comprised in the biomaterial of the invention is of between 1×10⁷ and 1×10¹⁵ CFU of probiotic bacteria per mg of BC, between 1×10⁸ and 1×10¹³ CFU of probiotic bacteria per mg of BC, between 1×10⁹ and 1×10¹² CFU of probiotic bacteria per mg of BC, between 1×10¹⁰ and 1×10¹¹ CFU of probiotic bacteria per mg of BC, between 5×10¹⁰ and 5×10¹¹ CFU of probiotic bacteria per mg of BC, between 7×10¹⁰ and 4×10¹¹ CFU of probiotic bacteria per mg of BC, between 8×10¹⁰ and 2×10¹¹ CFU of probiotic bacteria per mg of BC, between 8.5×10¹⁰ and 1.8×10¹¹ CFU of probiotic bacteria per mg of BC, between 8.7×10¹⁰ and 1.7×10¹¹ CFU of probiotic bacteria per mg of BC, between 8.7×10¹⁰ and 1.4×10¹¹ CFU of probiotic bacteria per mg of BC, between 9×10¹⁰ and 1.7×10¹¹ CFU of probiotic bacteria per mg of BC, between 9.2×10¹⁰ and 1.7×10¹¹ CFU of probiotic bacteria per mg of BC, between 9.2×10¹⁰ and 1.4×10¹¹ CFU of probiotic bacteria per mg of BC, between 1×10¹¹ and 1.2×10¹¹ CFU of probiotic bacteria per mg of BC, preferably between 8.5×10¹⁰ and 2×10¹¹ CFU of probiotic bacteria per mg of BC, more preferably between 8.7×10¹⁰ and 1.7×10¹¹ CFU of probiotic bacteria per mg of BC. In a preferred embodiment, the amount of probiotics comprised in the biomaterial of the invention is of between 8.5×10¹⁰ and 2×10¹¹ CFU of probiotic bacteria per mg of BC. In another preferred embodiment, the amount of probiotics comprised in the biomaterial of the invention is of between 1×10¹¹ and 1.2×10¹¹ CFU of probiotic bacteria per mg of BC.

As it will be understood by a skilled person, the probiotics comprised in the biomaterial of the invention are almost all entrapped in the BC matrix of the biomaterial of the invention. Thus, in a particular embodiment, the expression “the probiotics comprised in the biomaterial of the invention”, as used all along the specification, refers to the probiotics entrapped in the BC matrix of the biomaterial of the invention.

In a particular embodiment, the biomaterial of the invention is essentially free from bacteria that produce cellulose.

The expression “essentially free”, as used herein, refers to a biomaterial, which comprises less than 15%, 12%, 10%, 9%, 7%, 5%, 3%, 2%, 1.7%, 1.5%, 1.4%, 1.3%, 1.2%, 1.1%, 1%, 0.9%, 0.8%, 0.7%, 0.6% 0.5%, 0.4%, 0.3%, 0.2%, 0.1%, 0.09%, 0.085%, 0.08%, 0.07%, 0.06%, 0.005%, 0.04%, 0.03%, 0.02%, 0.01%, 0.009%, 0.008%, 0.007%, 0.006%, 0.005%, 0.004%, 0.003%, 0.002%, or 0.001% of cellulose producing bacteria with respect to the amount of probiotics comprised in the biomaterial.

The expression “essentially free”, as used herein, refers to a biomaterial, which comprises less than 15%, 12%, 10%, 9%, 7%, 5%, 3%, 2%, 1.7%, 1.5%, 1.4%, 1.3%, 1.2%, 1.1%, 1%, 0.9%, 0.8%, 0.7%, 0.6% 0.5%, 0.4%, 0.3%, 0.2%, 0.1%, 0.09%, 0.085%, 0.08%, 0.07%, 0.06%, 0.005%, 0.04%, 0.03%, 0.02%, 0.01%, 0.009%, 0.008%, 0.007%, 0.006%, 0.005%, 0.004%, 0.003%, 0.002%, or 0.001% of cellulose producing bacteria per weight unit of BC.

Methods for determining the amount of probiotics in the biomaterial of the invention or in a BC and of bacteria that produce cellulose with respect to the amount of probiotics in the biomaterial of the invention or in a BC are well known by an expert in the field. Non-limiting examples of methods allowing to determining the amount of probiotics in the biomaterial include those referred in the Materials and Methods “Quantification of immobilized probiotics” in the Examples below. Additional non-limiting examples of methods allowing counting the number of probiotics in the biomaterial or in a BC include those described below within the methods to count the number of bacteria that produce cellulose with respect to the number of probiotics in the biomaterial or BC, based on Field Emission Scanning Electron Microscopy (FESEM), on GRAM staining, or on a combination of both. The determination of the amount of bacteria that produce cellulose with respect to the amount of probiotics in the biomaterial or in a BC may be carried out by identifying probiotics and bacteria that produce cellulose using stains specific for each type of microorganism followed by counting the amount of bacteria that produce cellulose per number of probiotics identified in the biomaterial or BC of interest. Methods to identify and differentiate probiotics and bacteria that produce cellulose include those based on Field Emission Scanning Electron Microscopy (FESEM), on GRAM staining, or on a combination of both, as illustrated in Example 1 below. FESEM allows to differentiate both type of bacteria based on their different shape (See Example 1), and GRAM staining differentiates between Gram-negative bacteria (as most cellulose producing bacteria, such as bacteria from the genus Acetobacter, Gluconacetobacter, Komagataeibacter, Rhizobium, Agrobacterium, Enterobacter, Achromobacter, Azotobacter, Salmonella, Escherichia, Pseudomonas, Alcaligenis or Sarcina) and Gram-positive bacteria (as most probiotics, such as bacteria from the genus Lactobacillus, Bifidobacterium, Lactococcus, Streptococcus, enterococos, Pediococcus, Leuconostoc or Bacillus). Details about FESEM and GRAM staining methods are provided in Materials and Methods section in the Examples below. Methods allowing to count the number of bacteria that produce cellulose observed with any of said methods with respect to the amount of probiotics observed with any of said methods in the biomaterial or BC analyzed are well known by an expert in the field. They may simply consist on counting the amount of bacteria that produce cellulose identified and the number of probiotics identified with said methods in a specific surface unit of the biomaterial or BC analyzed and considered as a surface unit comprising a representative distribution of the different types of bacteria in the biomaterial or BC. The amount of bacteria that produce cellulose identified is then divided by the amount of probiotics identified. In case bacteria that produce cellulose are distributed in a different surface area of the biomaterial or BC than probiotics, the size of each of said surfaces is determined with respect to each other. The amount of bacteria that produce cellulose by surface unit where they are localized is determined, as well as the amount of probiotics by surface unit where they are localized with any of the methods referred above. The total amount of each of said type of bacteria is then normalized by the relative size of the surface area where they are localized as determined before. Then, the total amount of bacteria that produce cellulose so defined, is divided by the total amount of probiotics so defined in the biomaterial or in the bacteria cellulose analyzed.

In a particular embodiment, the bacterial cellulose has been produced by aerobic bacteria. As understood by a skilled person, said bacteria are bacteria that produce bacterial cellulose as defined above that in addition, are aerobic.

The expression “aerobic bacteria”, “obligate aerobic bacteria”, “aerobe”, or “obligate aerobe”, as used herein, refers to bacteria that require oxygen to grow or survive. In their metabolism of energy-containing compounds, aerobes require molecular oxygen as a terminal electron acceptor and cannot grow in its absence. In a particular embodiment, said organisms require an atmospheric oxygen concentration higher than 15%, 18%, 19%, 20%, 20.95%, 21%, 22%, preferably higher than 20%. Non-limiting examples of aerobic bacteria include bacteria from the genus Acetobacter, Gluconacetobacter, Komagataeibacter, Rhizobium, Agrobacterium, Achromobacter, Azotobacter, Pseudomonas or Alcaligenis.

In a particular embodiment, the aerobic bacteria that produce the BC of the biomaterial of the invention are from the genus Acetobacter, Gluconacetobacter, Komagataeibacter, Rhizobium, Agrobacterium, Achromobacter, Azotobacter, Pseudomonas, Alcaligenis or combinations thereof. In a preferred embodiment, the aerobic bacteria that produce the BC of the biomaterial of the invention are from the genus Acetobacter, Gluconacetobacter, Komagataeibacter or combinations thereof. In another preferred embodiment, aerobic bacteria that produce the BC of the biomaterial of the invention are for the genus Acetobacter. In a particular embodiment, aerobic bacteria that produce the BC of the biomaterial of the invention are from the genus Gluconacetobacter. In another particular embodiment, aerobic bacteria that produce the BC of the biomaterial of the invention are from the genus Komagataeibacter

In a particular embodiment, bacteria from a genus specified above are from any of the species of said genus.

In a particular embodiment, the aerobic bacteria that produce the BC of the biomaterial of the invention from the genus Acetobacter are from the species A. xylinum, A. nitrogenifigens, A. orientalis or combinations thereof. In a preferred embodiment, the bacteria from the genus Acetobacter are from the species A. xylinum, preferably from the strain deposited at the Colección Española de Cultivos Tipo (CECT) with accession number CECT 473.

As well known by an expert in the art, Acetobacter xylinum, Gluconacetobacter xylinum and Komagataeibacter xylinum are often used interchangeably. Thus, in some embodiments, Acetobacter xylinum, refers to Gluconacetobacter xylinum. In other embodiments, A. xylinum refers to Komagataeibacter xylinum.

A. xylinum CECT 473 is a strain freely available from Colección Española de Cultivos Tipo.

In a particular embodiment, the aerobic bacteria that produce the BC of the biomaterial of the invention from the Gluconacetobacter are from the species G. hansenii, G. swingsii, G. sacchari, G. kombuchae, G. entanii, G. persimmonis, G. sucrofermentans or combinations thereof.

In a particular embodiment, the aerobic bacteria that produce the BC of the biomaterial of the invention from the genus Komagataeibacter are from the species K. europaeus, K. medellinensis, K. intermedius, K. rhaeticus, K. kakiaceti, K. oboediens, K. nataicola, K. saccharivorans, K. maltaceti, or combinations thereof.

In a particular embodiment, the bacterial cellulose has been produced by anaerobic bacteria. As understood by a skilled person, said bacteria are bacteria that produce bacterial cellulose as defined above that in addition, are anaerobic.

The expression “anaerobic bacteria”, as used herein refers to bacteria that cannot grow in the presence of oxygen. Their metabolism frequently is a fermentative type in which they reduce available organic compounds to various end products such as organic acids and alcohols. Oxygen tolerance varies between species, some are capable of surviving in up to 8% atmospheric oxygen concentration, others lose viability unless the oxygen concentration in the atmosphere is less than 0.5%. Thus, in a particular embodiment, the anaerobic bacteria require an atmospheric oxygen concentration lower than 8%, 7%, 6%; 5%, 4%, 3%, 2%, 1%, 0.9%, 0.7%, 0.5%, 0.4%, 0.3%, 0.2%, 0.1%, preferably lower than 8%.

In a particular embodiment, the anaerobic bacteria that produce the BC of the biomaterial of the invention are from the genus Sarcina, preferably from the species S. ventriculi.

In a particular embodiment, the bacterial cellulose has been produced by facultative anaerobic bacteria. As understood by a skilled person, said bacteria are bacteria that produce bacterial cellulose as defined above that in addition, are facultative anaerobes.

The expression “facultative anaerobic bacteria”, as used herein, refers to bacteria that can grow or survive in the presence or absence of oxygen, because they can metabolize energy aerobically or anaerobically. They preferentially utilize oxygen as a terminal electron acceptor, but also can metabolize in the absence of oxygen by reducing other compounds. Thus, in the presence of oxygen facultative anaerobic bacteria metabolize energy aerobically and in the absence of oxygen they metabolize energy anaerobically. Thus, facultative anaerobic bacteria can grow in any of the oxygen concentrations indicated above in the definition of “aerobic bacteria” and of “anaerobic bacteria”.

In a particular embodiment, the facultative anaerobic bacteria that produce BC of the biomaterial of the invention are from the genus Enterobacter, Salmonella, Escherichia, or combinations thereof.

In a particular embodiment, the bacterial cellulose has been produced by microaerophiles. As understood by a skilled person, said bacteria are bacteria that produce bacterial cellulose as defined above that in addition, are microaerophiles.

The term “microaerophile”, as used herein, refers to bacteria that metabolize energy aerobically, and not anaerobically, although normal oxygen concentrations are toxic for these bacteria. Microaerophiles thus grow in oxygen atmospheric concentrations between 2-10%.

In particular embodiment, they grow under atmospheric oxygen concentrations of between 0.5% and 21%, 1% and 18%, 1.5% and 15%, 2% and 10%, 5% and 10%, 5% and 8%, preferably between 2% and 10%. In another particular embodiment, Microaerophiles require an atmospheric oxygen concentration of between 0.5% and 21%, 1% and 18%, 1.5% and 15%, 2% and 10%, 5% and 10%, 5% and 8%, preferably between 2% and 10%.

In a particular embodiment, the probiotics comprised in the biomaterial of the invention are facultative anaerobic bacteria. In another particular embodiment, the probiotics comprised in the biomaterial of the invention are aerotolerant anaerobic bacteria. In another particular embodiment, the probiotics comprised in the biomaterial of the invention are facultative anaerobic bacteria or aerotolerant anaerobic bacteria. In a preferred embodiment, the probiotics comprised in the biomaterial of the invention are facultative anaerobic bacteria and/or aerotolerant anaerobic bacteria.

The expression “aerotolerant anaerobes” or “aerotolerant anaerobic bacteria” as used herein, refers to bacteria that metabolize energy anaerobically and thus do not require oxygen to grow or survive, but that are not poisoned by oxygen, i.e. they tolerate the presence of oxygen in the atmosphere. In a particular embodiment, aerotolerant anaerobes grow with an oxygen concentration in the atmosphere lower than 21%, 18%, 15%, 12%, 10%, 8,%, 7%, 6%, 5%, 4%, 4%, 3%, 2%, 1%, 0.5%, 0.25%, 0.2%, 0.1%, 0.9%, 0.5%, 0.25%, preferably lower 10%. In preferred embodiment, aerotolerant anaerobes grow with an oxygen concentration in the atmosphere of between 0.5% and 21%, 1% and 18%, 1.5% and 15%, 2% and 10%, 5% and 10%, 5% and 8%, preferably of between 2% and 10%. In another particular embodiment, they require an atmospheric oxygen concentration lower than 21%, 18%, 15%, 12%, 10%, 8,%, 7%, 6%, 5%, 4%, 4%, 3%, 2%, 1%, 0.5%, 0.25%, 0.2%, 0.1%, 0.9%, 0.5%, 0.25%, preferably lower 10%. In another embodiment, aerotolerant anaerobes require an oxygen concentration in the atmosphere of between In preferred embodiment, they grow with an oxygen concentration in the atmosphere of between 0.5% and 21%, 1% and 18%, 1.5% and 15%, 2% and 10%, 5% and 10%, 5% and 8%, preferably of between 2% and 10%.

In a particular embodiment, probiotics comprised in the biomaterial of the invention as described herein are from the genus Lactobacillus, Bifidobacterium, Lactococcus, Streptococcus, Enterococcus, Pediococcus, Leuconostoc, Bacillus, Escherichia or combinations thereof. In a preferred embodiment, the probiotics comprised in the biomaterial of the invention as described herein are from the genus Lactobacillus, Bifidobacterium, Lactococcus, Streptococcus, or combinations thereof. In another preferred embodiment, they are from the genus Lactobacillus. In another preferred embodiment, they are from the genus Bifidobacterium.

In some embodiments, probiotics comprised in the biomaterial of the invention as defined herein are from a species from one of the genus mentioned in the previous embodiment. In other embodiments, they are from several species from one genera indicated in the aforementioned embodiments. In other embodiments, the probiotics comprised in the biomaterial of the invention are from several species from at least two genera selected from those indicated in the aforementioned embodiments.

In a particular embodiment, probiotics comprised in the biomaterial of the invention that are from the genus Lactobacillus are from the species L. fermentum, L. gasseri, L. acidophilus, L. plantarum., L. rhamnosus, L. casei, L. johnsonii, L. delbrueckii, L. salivarus, or combinations thereof. In a preferred embodiment, they are from the species L. fermemtum. In another preferred embodiment, the probiotics comprised in the biomaterial of the invention are from the species L. gasseri.

In a preferred embodiment, probiotics comprised in the biomaterial of the invention are from the genus Lactobacillus, preferably from the species L. fermentum, L. gasseri, L. acidophilus, L. plantarum., L. rhamnosus, L. casei, L. johnsonii, L. delbrueckii, L. salivarus, or combinations thereof. In a preferred embodiment, they are form the species L. fermemtum. In another preferred embodiment, the probiotics comprised in the biomaterial of the invention are from the species L. gasseri.

In a particular embodiment, the probiotics comprised in the biomaterial of the invention that are from the species L. acidophilus are from the strain CECT 903.

L. acidophilus CECT 903 is a strain that is freely available from Colección Española de Cultivos Tipo.

In another particular embodiment, the probiotics comprised in the biomaterial of the invention that are from the species L. plantarum are from the strain CECT 220.

L. plantarum CECT 220 is a strain that is freely available from Colección Española de Cultivos Tipo.

In another particular embodiment, the probiotics comprised in the biomaterial of the invention that are from the species L. rhamnosus are from the strain CECT 278.

L. rhamnosus CECT 278 is a strain freely available from Colección Española de Cultivos Tipo

In a particular embodiment, the probiotics comprised in the biomaterial of the invention that are from the genus Bifidobacterium are from the species B. breve, B. longum, B. animalis, B. infantum, B. animalis, lactis, Bifidobacterium thermophilum, Bifidobacterium boum, Bifidobacterium minimum, Bifidobacterium pyschraerophilum or combinations thereof. In another particular embodiment, the comprised in the biomaterial of the invention that are from the genus Bifidobacterium are from the species B. breve, B. longum, B. animalis, B. infantum, B. animalis, or combinations thereof. In a preferred embodiment, they are from the species B. breve. In a preferred embodiment, the probiotics comprised in the biomaterial of the invention are from the species B. breve.

In a particular embodiment, the probiotics comprised in the biomaterial of the invention are from the genus Bifidobacterium, preferably from the species B. breve, B. longum, B. animalis, B. infantum, B. animalis, lactis, Bifidobacterium thermophilum, Bifidobacterium boum, Bifidobacterium minimum, Bifidobacterium pyschraerophilum or combinations thereof. In another embodiment, the probiotics comprised in the biomaterial of the invention are from the genus Bifidobacterium, preferably from the species B. breve, B. longum, B. animalis, B. infantum, B. animalis, or combinations thereof. In a preferred embodiment, they are from the species B. breve. In a preferred embodiment, the probiotics comprised in the biomaterial of the invention are from the species B. breve.

In a particular embodiment, the probiotics comprised in the biomaterial of the invention that are from the species Streptococcus are from the species S. thermophiles.

In a particular embodiment, probiotics comprised in the biomaterial of the invention that are facultative anaerobic bacteria are from the genus Lactobacillus, Bifidobacterium, Lactococcus, Streptococcus, Enterococcus, Pediococcus, Leuconostoc, Bacillus, Escherichia or combinations thereof. In another particular embodiment, the probiotics comprised in the biomaterial of the invention that are anaerobic facultative bacteria are from the genus Lactobacillus, Bifidobacterium, Lactococcus, Streptococcus, or combinations thereof. In another embodiment, probiotics comprised in the biomaterial of the invention that are anaerobic facultative bacteria are from the genus Lactobacillus, Lactococcus, Streptococcus, Enterococcus, Pediococcus, Leuconostoc, Bacillus, Escherichia or combinations thereof. In another embodiment, probiotics comprised in the biomaterial of the invention that are anaerobic facultative bacteria are from the genus Lactobacillus, Lactococcus, Streptococcus, or combinations thereof. In a preferred embodiment, probiotics comprised in the biomaterial of the invention that are facultative anaerobic bacteria are from the genus Lactobacillus.

In another particular embodiment, probiotics comprised in the biomaterial of the invention that are aerotolorant anaerobes are from the genus Bifidobacterium. In a particular embodiment, the probiotics comprised in the biomaterial of the invention that are facultative anaerobic bacteria that are from the genus Lactobacillus are from the species L. fermentum, L. gasseri, L. acidophilus, L. plantarum., L. rhamnosus, L. casei, L. johnsonii, L. delbrueckii, L. salivarus, or combinations thereof. In preferred embodiment, they are form the species L. fermemtum. In another preferred embodiment, the probiotics comprised in the biomaterial of the invention that are facultative anaerobic bacteria are from the species L. gasseri.

In a preferred embodiment, probiotics comprised in the biomaterial of the invention are facultative anaerobic bacteria from the genus Lactobacillus, preferably from the species L. fermentum, L. gasseri, L. acidophilus, L. plantarum., L. rhamnosus, L. casei, L. johnsonii, L. delbrueckii, L. salivarus, or combinations thereof. In preferred embodiment, probiotics comprised in the biomaterial of the invention are facultative anaerobic bacteria from the species L. fermemtum. In another preferred embodiment, probiotics comprised in the biomaterial of the invention are facultative anaerobic bacteria from the species L. gasseri. In a particular embodiment, the probiotics comprised in the biomaterial of the invention that are facultative anaerobic bacteria that are from the species L. acidophilus are from the strain CECT 903.

In another particular embodiment, the probiotics comprised in the biomaterial of the invention that are facultative anaerobic bacteria that are from the species L. plantarum are from the strain CECT 220.

In another particular embodiment, the probiotics comprised in the biomaterial of the invention that are facultative anaerobic bacteria that are from the species L. rhamnosus are from the strain CECT 278.

In a particular embodiment, the probiotics comprised in the biomaterial of the invention that are facultative anaerobic bacteria that are from the genus Bifidobacterium are from the species B. breve, B. longum, B. animalis, B. infantum, B. animalis, lactis, Bifidobacterium thermophilum, Bifidobacterium boum, Bifidobacterium minimum, Bifidobacterium pyschraerophilum or combinations thereof. In another particular embodiment, the probiotics comprised in the biomaterial of the invention that are facultative anaerobic bacteria that are from the genus Bifidobacterium are from the species B. breve, B. longum, B. animalis, B. infantum, B. animalis, or combinations thereof. In another particular embodiment, the probiotics comprised in the biomaterial of the invention that are facultative anaerobic bacteria that are from the genus Bifidobacterium are from the species B. breve, B. longum, B. animalis, B. infantum, B. animalis, or combinations thereof. In a preferred embodiment, the probiotics comprised in the biomaterial of the invention that are facultative anaerobic bacteria are from the species B. breve.

In a particular embodiment, the probiotics comprised in the biomaterial of the invention that are facultative anaerobic bacteria that are from the species Lactococcus are from the species L. lactis.

In a particular embodiment, the probiotics comprised in the biomaterial of the invention that are facultative anaerobic bacteria that are from the species Streptococcus are from the species S. thermophiles.

In a particular embodiment, the probiotics comprised in the biomaterial of the invention that are aerotolerant anaerobes are from the genus Bifidobacterium.

In a particular embodiment, the probiotics comprised in the biomaterial of the invention that are aerotolerant anaerobes that are from the genus Bifidobacterium are from the species B. breve, B. longum, B. animalis, B. infantum, B. animalis, lactis, Bifidobacterium thermophilum, Bifidobacterium boum, Bifidobacterium minimum, Bifidobacterium pyschraerophilum or combinations thereof. In another particular embodiment, the probiotics comprised in the biomaterial of the invention that are aerotolerant anaerobes are from the genus Bifidobacterium animalis subsp. lactis, Bifidobacterium thermophilum, Bifidobacterium boum, Bifidobacterium minimum, Bifidobacterium pyschraerophilum, and combinations thereof. In another particular embodiment, the probiotics comprised in the biomaterial of the invention that are aerotolerant anaerobes are from the species B. breve, B. longum, B. animalis, B. infantum, B. animalis or combinations thereof. In a preferred embodiment, the probiotics comprised in the biomaterial of the invention that are aerotolrant anaerobes are from the species B. breve.

In a particular embodiment, the probiotics comprised in the biomaterial of the invention are aerotolerant anaerobes from the genus Bifidobacterium, preferably from the species B. breve, B. longum, B. animalis, B. infantum, B. animalis, lactis, Bifidobacterium thermophilum, Bifidobacterium boum, Bifidobacterium minimum, Bifidobacterium pyschraerophilum or combinations thereof. In another particular embodiment, probiotics comprised in the biomaterial of the invention are aerotolerant anaerobes from the genus Bifidobacterium, preferably from the species Bifidobacterium animalis subsp. lactis, Bifidobacterium thermophilum, Bifidobacterium boum, Bifidobacterium minimum, Bifidobacterium pyschraerophilum, and combinations thereof. In another particular embodiment, probiotics comprised in the biomaterial of the invention are aerotolerant anaerobes from the genus Bifidobacterium, preferably from the species B. breve, B. longum, B. animalis, B. infantum, B. animalis or combinations thereof. In a preferred embodiment, probiotics comprised in the biomaterial of the invention are aerotolerant anaerobes from the species B. breve.

In particular embodiment, probiotics comprised in the biomaterial of the invention that are facultative anaerobic bacteria and/or aerotolerant anaerobes are found at a concentration with respect to the content of BC as defined in the embodiments above for the amount of probiotics comprised in the biomaterial of the invention.

In another particular embodiment, the facultative anaerobic probiotics comprised in the biomaterial according to the invention are found at a concentration with respect to the content of BC as defined in the embodiments above for the amount of probiotics comprised in the biomaterial of the invention. In a preferred embodiment, the amount of facultative anaerobic probiotics comprised in the biomaterial of the invention is of about 8.7×10¹⁰ CFU of probiotic bacteria per mg BC, preferably about 9.2×10¹⁰ CFU of probiotic bacteria per mg BC, more preferably about 1×10¹¹ CFU of probiotic bacteria per mg of BC, yet more preferably about 1.2×10¹¹ CFU of probiotic bacteria per mg of BC, even yet more preferably about 1.4×10¹¹ CFU of probiotic bacteria per mg of BC, even more preferably about 1.7×10¹¹ CFU of probiotic bacteria per mg of BC. In a preferred embodiment, it is of about 1.2×10¹¹ CFU of probiotic bacteria per mg of BC. In another preferred embodiment, the amount of facultative anaerobic probiotics comprised in the biomaterial of the invention is of 1×10¹¹ CFU of probiotic bacteria per mg of BC. In a another preferred embodiment, it is of at least 8.7×10¹⁰ CFU of probiotic bacteria per mg of BC, preferably of at least 9.2×10¹⁰ CFU of probiotic bacteria per mg of BC, more preferably at least 1×10¹¹ CFU of probiotic bacteria per mg of BC, yet more preferably at least 1.2×10¹¹ CFU of probiotic bacteria per mg of BC, even more preferably of at least 1.4×10¹¹ CFU of probiotic bacteria per mg of BC, even yet more preferably at least 1.7×10¹¹ CFU of probiotic bacteria per mg of BC. In a preferred embodiment, the amount of facultative anaerobic probiotics comprised in the biomaterial of the invention is of at least 1.2×10¹¹ CFU of probiotic bacteria per mg of BC. In another preferred embodiment, the amount of facultative anaerobic probiotics comprised in the biomaterial of the invention is of at least 1×10¹¹ CFU of probiotic bacteria per mg of BC. In another preferred embodiment, the amount of facultative anaerobic probiotics comprised in the biomaterial of the invention is of between 8.5×10¹⁰ CFU of probiotic bacteria per mg of BC and 2×10¹¹ CFU of probiotic bacteria per mg of BC. In another preferred embodiment, the amount of facultative anaerobic probiotics comprised in the biomaterial of the invention is of between 1×10¹¹ and 1.2×10¹¹ CFU of probiotic bacteria per mg of BC.

In a particular embodiment, the aerotolerant anaerobic probiotics comprised in the biomaterial of the invention are found at a concentration with respect to the content of BC as defined in the embodiments above for the amount of probiotics comprised in the biomaterial of the invention. In a preferred embodiment, the amount of aerotolerant anaerobic probiotics comprised in the biomaterial of the invention is of about 8.7×10¹⁰ CFU of probiotic bacteria per mg BC, preferably about 9.2×10¹⁰ CFU of probiotic bacteria per mg BC, more preferably about 1×10¹¹ CFU of probiotic bacteria per mg of BC, yet more preferably about 1.2×10¹¹ CFU of probiotic bacteria per mg of BC, even yet more preferably about 1.4×10¹¹ CFU of probiotic bacteria per mg of BC, even more preferably about 1.7×10¹¹ CFU of probiotic bacteria per mg of BC. In a preferred embodiment, the amount of aerotolerant anaerobic probiotics comprised in the biomaterial of the invention is of about 1.2×10¹¹ CFU of probiotic bacteria per mg of BC. In another preferred embodiment, the amount of aerotolerant anaerobic probiotics comprised in the biomaterial of the invention is of 1×10¹¹ CFU of probiotic bacteria per mg of BC. In a another preferred embodiment, it is of at least 8.7×10¹⁰ CFU of probiotic bacteria per mg of BC, preferably of at least 9.2×10¹⁰ CFU of probiotic bacteria per mg of BC, more preferably at least 1×10¹¹ CFU of probiotic bacteria per mg of BC, even more preferably at least 1.2×10¹¹ CFU of probiotic bacteria per mg of BC, yet more preferably at least 1.4×10¹¹ CFU of probiotic bacteria per mg of BC, even yet more preferably of at least 1.7×10¹¹ CFU of probiotic bacteria per mg of BC. In a preferred embodiment, the amount of aerotolerant anaerobic probiotics comprised in the biomaterial of the invention is of at least 1.2×10¹¹ CFU of probiotic bacteria per mg of BC. In a preferred embodiment, the amount of aerotolerant anaerobic probiotics comprised in the biomaterial of the invention is of at least 1×10¹¹ CFU of probiotic bacteria per mg of BC. In another preferred embodiment, the amount of aerotolerant anaerobic probiotics comprised in the biomaterial of the invention is of between 8.5×10¹⁰ CFU of probiotic bacteria per mg of BC and 2×10¹¹ CFU of probiotic bacteria per mg of BC. In another preferred embodiment, the amount of aerotolerant anaerobic probiotics comprised in the biomaterial of the invention is of between 1×10¹¹ and 1.2×10¹¹ CFU of probiotic bacteria per mg of BC.

In particular embodiment, the probiotics comprised in the biomaterial of the invention as defined herein are preferably anaerobic facultative bacteria, are from the genus Lactobacillus, preferably from the species L. fermentum, and the amount of said probiotics comprised in the biomaterial of the invention is of about 5×10¹⁰, 7×10¹⁰, 9×10¹⁰, 1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.5×10¹¹, 2.7×10¹¹, 3×10¹¹, 3.5×10¹¹, 4×10¹¹, 4.5×10¹¹, 5×10¹¹, 6×10¹¹, 7×10¹¹, 8×10¹¹, 9×10¹¹, 1×10¹², 2×10¹² CFU of probiotics per mg BC, preferably about 1×10¹¹ CFU of probiotics per mg BC, more preferably about 1.2×10¹¹ CFU of probiotics per mg BC, yet more preferably about 1.4×10¹¹ CFU of probiotics per mg BC, even yet more preferably about 1.7×10¹¹ CFU of probiotics per mg BC. In a preferred embodiment, the amount of said probiotics comprised in the biomaterial of the invention is of about 1.2×10¹¹ CFU of probiotics per mg BC. In another preferred embodiment, the amount of said probiotics comprised in the biomaterial of the invention is of about 1×10¹¹ CFU of probiotics per mg BC.

In particular embodiment, the probiotics comprised in the biomaterial of the invention as defined herein are preferably anaerobic facultative bacteria, are from the genus Lactobacillus, preferably from the species L. fermentum, and the amount of said probiotics comprised in the biomaterial is of at least any of the amounts indicated in the embodiment just above.

In particular embodiment, probiotics comprised in the biomaterial of the invention as defined herein are preferably anaerobic facultative bacteria, are from the genus Lactobacillus, preferably form the species L. fermentum, and the amount of said probiotics comprised in the biomaterial is of between 1×10⁷ and 1×10¹⁶ CFU of probiotic bacteria per mg of BC, between 1×10⁸ and 1×10¹³ CFU of probiotic bacteria per mg of BC, between 1×10⁹ and 1×10¹² CFU of probiotic bacteria per mg of BC, between 1×10¹⁰ and 1×10¹¹ CFU of probiotic bacteria per mg of BC, between 5×10¹⁰ and 5×10¹¹ CFU of probiotic bacteria per mg of BC, between 7×10¹⁰ and 4×10¹¹ CFU of probiotic bacteria per mg of BC, between 8×10¹⁰ and 2×10¹¹ CFU of probiotic bacteria per mg of BC, between 8.5×10¹⁰ and 1.8×10¹¹ CFU of probiotic bacteria per mg of BC, between 8.7×10¹⁰ and 1.7×10¹¹ CFU of probiotic bacteria per mg of BC, between 8.7×10¹⁰ and 1.4×10¹¹ CFU of probiotic bacteria per mg of BC, between 9×10¹⁰ and 1.7×10¹¹ CFU of probiotic bacteria per mg of BC, between 9.2×10¹⁰ and 1.7×10¹¹ CFU of probiotic bacteria per mg of BC, between 9.2×10¹⁰ and 1.4×10¹¹ CFU of probiotic bacteria per mg of BC, between 1×10¹¹ and 1.2×10¹¹ CFU of probiotic bacteria per mg of BC, preferably between 8.5×10¹⁰ and 2×10¹¹ CFU of probiotic bacteria per mg of BC, more preferably between 8.7×10¹⁰ and 1.7×10¹¹ CFU of probiotic bacteria per mg of BC. In a preferred embodiment, the amount of said probiotics comprised in the biomaterial of the invention is of between 8.5×10¹⁰ CFU of probiotic bacteria per mg of BC and 2×10¹¹ CFU of probiotic bacteria per mg of BC. In another preferred embodiment, the amount of said probiotics comprised in the biomaterial of the invention is of between 1×10¹¹ and 1.2×10¹¹ CFU of probiotic bacteria per mg of BC In particular embodiment, probiotics comprised in the biomaterial of the invention as defined herein are preferably anaerobic facultative bacteria, are from the genus Lactobacillus, preferably from the species L. gasseri, and the amount of said probiotics comprised in the biomaterial is of about 5×10¹⁰, 7×10¹⁰, 9×10¹⁰, 1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.5×10¹¹, 2.7×10¹¹, 3×10¹¹, 3.5×10¹¹, 4×10¹¹, 4.5×10¹¹, 5×10¹¹, 6×10¹¹, 7×10¹¹, 8×10¹¹, 9×10¹¹, 1×10¹², 2×10¹² of probiotics per mg BC, preferably about 8.7×10¹⁰ of probiotics per mg BC, more preferably about 9.2×10¹⁰ of probiotics per mg BC, yet more preferably about 1×10¹⁰ of probiotics per mg BC, even yet more preferably 1.2×10¹⁰ of probiotics per mg BC. In a preferred embodiment, the amount of said probiotics comprised in the biomaterial of the invention is of about 1.2×10¹¹ CFU of probiotics per mg BC. In another preferred embodiment, the amount of said probiotics comprised in the biomaterial of the invention is of about 1×10¹¹ CFU of probiotics per mg BC.

In particular embodiment, the probiotics comprised in the biomaterial of the invention as defined herein are preferably anaerobic facultative bacteria, are from the genus Lactobacillus, preferably from the species L. gasseri, and the amount of said probiotics comprised in the biomaterial is of at least any of those indicated in the embodiment just above.

In particular embodiment, the probiotics comprised in the biomaterial of the invention as defined herein are preferably anaerobic facultative bacteria, are from the genus Lactobacillus, preferably from the species L. gasseri, and the amount of said probiotics in the biomaterial is of between 1×10⁷ and 1×10¹⁶ CFU of probiotic bacteria per mg of BC, between 1×10⁸ and 1×10¹³ CFU of probiotic bacteria per mg of BC, between 1×10⁹ and 1×10¹² CFU of probiotic bacteria per mg of BC, between 1×10¹⁰ and 1×10¹¹ CFU of probiotic bacteria per mg of BC, between 5×10¹⁰ and 5×10¹¹ CFU of probiotic bacteria per mg of BC, between 7×10¹⁰ and 4×10¹¹ CFU of probiotic bacteria per mg of BC, between 8×10¹⁰ and 2×10¹¹ CFU of probiotic bacteria per mg of BC, between 8.5×10¹⁰ and 1.8×10¹¹ CFU of probiotic bacteria per mg of BC, between 8.7×10¹⁰ and 1.7×10¹¹ CFU of probiotic bacteria per mg of BC, between 8.7×10¹⁰ and 1.4×10¹¹ CFU of probiotic bacteria per mg of BC, between 9×10¹⁰ and 1.7×10¹¹ CFU of probiotic bacteria per mg of BC, between 9.2×10¹⁰ and 1.7×10¹¹ CFU of probiotic bacteria per mg of BC, between 9.2×10¹⁰ and 1.4×10¹¹ CFU of probiotic bacteria per mg of BC, between 1×10¹¹ and 1.2×10¹¹ CFU of probiotic bacteria per mg of BC, preferably between 8.5×10¹⁰ and 2×10¹¹ CFU of probiotic bacteria per mg of BC, more preferably between 8.7×10¹⁰ and 1.7×10¹¹ CFU of probiotic bacteria per mg of BC. In a preferred embodiment, the amount of said probiotics comprised in the biomaterial of the invention is of between 8.5×10¹⁰ CFU of probiotic bacteria per mg of BC and 2×10¹¹ CFU of probiotic bacteria per mg of BC. In another preferred embodiment, the amount of said probiotics comprised in the biomaterial of the invention is of between 1×10¹¹ and 1.2×10¹¹ CFU of probiotic bacteria per mg of BC.

In a particular embodiment, the probiotics comprised in the biomaterial of the invention as defined herein are preferably anaerobic facultative bacteria, are from the genus Bifidobacterium, preferably from the species B. breve, and the amount of said probiotics comprised in the biomaterial is of about 5×10¹⁰, 7×10¹⁰, 9×10¹⁰, 1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.5×10¹¹, 2.7×10¹¹, 3×10¹¹, 3.5×10¹¹, 4×10¹¹, 4.5×10¹¹, 5×10¹¹, 6×10¹¹, 7×10¹¹, 8×10¹¹, 9×10¹¹, 1×10¹², 2×10¹² CFU of probiotics per mg BC, preferably about 8.7×10¹⁰ of probiotics per mg BC, more preferably about 9.2×10¹⁰ of probiotics per mg BC about 1×10¹¹ CFU of probiotics per mg BC, even more preferably about 1.2×10¹¹ CFU of probiotics per mg BC, yet more preferably about 1.4×10¹¹ CFU of probiotics per mg BC, even yet more preferably about 1.7×10¹¹ CFU of probiotics per mg BC. In a preferred embodiment, the amount of said probiotics comprised in the biomaterial of the invention is of about 1.2×10¹¹ CFU of probiotics per mg BC. In another preferred embodiment, the amount of said probiotics comprised in the biomaterial of the invention is of about 1×10¹¹ CFU of probiotics per mg BC.

In particular embodiment, the probiotics comprised in the biomaterial of the invention as defined herein are preferably anaerobic facultative bacteria, are from the genus Bifidobacterium, preferably from the species B. breve, and the amount of said probiotics comprised in the biomaterial is of at least any of the amounts indicated in the embodiment just above.

In particular embodiment, probiotics comprised in the biomaterial of the invention as defined herein are preferably anaerobic facultative bacteria, are from the genus Bifidobacterium, preferably from the species B. breve, and the amount of said probiotics comprised in the biomaterial is of between 1×10⁷ and 1×10¹⁶ CFU of probiotic bacteria per mg of BC, between 1×10⁸ and 1×10¹³ CFU of probiotic bacteria per mg of BC, between 1×10⁹ and 1×10¹² CFU of probiotic bacteria per mg of BC, between 1×10¹⁰ and 1×10¹¹ CFU of probiotic bacteria per mg of BC, between 5×10¹⁰ and 5×10¹¹ CFU of probiotic bacteria per mg of BC, between 7×10¹⁰ and 4×10¹¹ CFU of probiotic bacteria per mg of BC, between 8×10¹⁰ and 2×10¹¹ CFU of probiotic bacteria per mg of BC, between 8.5×10¹⁰ and 1.8×10¹¹ CFU of probiotic bacteria per mg of BC, between 8.7×10¹⁰ and 1.7×10¹¹ CFU of probiotic bacteria per mg of BC, between 8.7×10¹⁰ and 1.4×10¹¹ CFU of probiotic bacteria per mg of BC, between 9×10¹⁰ and 1.7×10¹¹ CFU of probiotic bacteria per mg of BC, between 9.2×10¹⁰ and 1.7×10¹¹ CFU of probiotic bacteria per mg of BC, between 9.2×10¹⁰ and 1.4×10¹¹ CFU of probiotic bacteria per mg of BC, between 1×10¹¹ and 1.2×10¹¹ CFU of probiotic bacteria per mg of BC, preferably between 8.5×10¹⁰ and 2×10¹¹ CFU of probiotic bacteria per mg of BC, more preferably between 8.7×10¹⁰ and 1.7×10¹¹ CFU of probiotic bacteria per mg of BC. In a preferred embodiment, the amount of said probiotics comprised in the biomaterial of the invention is of between 8.5×10¹⁰ CFU of probiotic bacteria per mg of BC and 2×10¹¹ CFU of probiotic bacteria per mg of BC. In another preferred embodiment, the amount of said probiotics comprised in the biomaterial of the invention is of between 1×10¹¹ and 1.2×10¹¹ CFU of probiotic bacteria per mg of BC.

In a particular embodiment, the probiotics comprised in the biomaterial of the invention as defined herein are preferably aerotolerant facultative bacteria, are from the genus Bifidobacterium, preferably from the species B. breve, and the amount of said probiotics comprised in the biomaterial is of about 5×10¹⁰, 7×10¹⁰, 9×10¹⁰, 1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.5×10¹¹, 2.7×10¹¹, 3×10¹¹, 3.5×10¹¹, 4×10¹¹, 4.5×10¹¹, 5×10¹¹, 6×10¹¹, 7×10¹¹, 8×10¹¹, 9×10¹¹, 1×10¹², 2×10¹² CFU of probiotics per mg BC, preferably about 8.7×10¹⁰ of probiotics per mg BC, more preferably about 9.2×10¹⁰ of probiotics per mg BC about 1×10¹¹ CFU of probiotics per mg BC, even more preferably about 1.2×10¹¹ CFU of probiotics per mg BC, yet more preferably about 1.4×10¹¹ CFU of probiotics per mg BC, even yet more preferably about 1.7×10¹¹ CFU of probiotics per mg BC. In a preferred embodiment, the amount of said probiotics comprised in the biomaterial of the invention is of about 1.2×10¹¹ CFU of probiotics per mg BC. In another preferred embodiment, the amount of said probiotics comprised in the biomaterial of the invention is of about 1×10¹¹ CFU of probiotics per mg BC.

In particular embodiment, the probiotics comprised in the biomaterial of the invention as defined herein are preferably aerotolerant facultative bacteria, are from the genus Bifidobacterium, preferably from the species B. breve, and the amount of said probiotics comprised in the biomaterial is of at least any of the amounts indicated in the embodiment just above.

In particular embodiment, probiotics comprised in the biomaterial of the invention as defined herein are preferably aerotolerant facultative bacteria, are from the genus Bifidobacterium, preferably from the species B. breve, and the amount of said probiotics comprised in the biomaterial is of between 1×10⁷ and 1×10¹⁶ CFU of probiotic bacteria per mg of BC, between 1×10⁸ and 1×10¹³ CFU of probiotic bacteria per mg of BC, between 1×10⁹ and 1×10¹² CFU of probiotic bacteria per mg of BC, between 1×10¹⁰ and 1×10¹¹ CFU of probiotic bacteria per mg of BC, between 5×10¹⁰ and 5×10¹¹ CFU of probiotic bacteria per mg of BC, between 7×10¹⁰ and 4×10¹¹ CFU of probiotic bacteria per mg of BC, between 8×10¹⁰ and 2×10¹¹ CFU of probiotic bacteria per mg of BC, between 8.5×10¹⁰ and 1.8×10¹¹ CFU of probiotic bacteria per mg of BC, between 8.7×10¹⁰ and 1.7×10¹¹ CFU of probiotic bacteria per mg of BC, between 8.7×10¹⁰ and 1.4×10¹¹ CFU of probiotic bacteria per mg of BC, between 9×10¹⁰ and 1.7×10¹¹ CFU of probiotic bacteria per mg of BC, between 9.2×10¹⁰ and 1.7×10¹¹ CFU of probiotic bacteria per mg of BC, between 9.2×10¹⁰ and 1.4×10¹¹ CFU of probiotic bacteria per mg of BC, between 1×10¹¹ and 1.2×10¹¹ CFU of probiotic bacteria per mg of BC, preferably between 8.5×10¹⁰ and 2×10¹¹ CFU of probiotic bacteria per mg of BC, more preferably between 8.7×10¹⁰ and 1.7×10¹¹ CFU of probiotic bacteria per mg of BC. In a preferred embodiment, the amount of said probiotics comprised in the biomaterial of the invention is of between 8.5×10¹⁰ CFU of probiotic bacteria per mg of BC and 2×10¹¹ CFU of probiotic bacteria per mg of BC. In another preferred embodiment, the amount of said probiotics comprised in the biomaterial of the invention is of between 1×10¹¹ and 1.2×10¹¹ CFU of probiotic bacteria per mg of BC.

In a particular embodiment, the probiotics comprised in the biomaterial of the invention are a population of probiotics, comprising facultative anaerobic bacteria and aerotolerant anaerobic bacteria. In a preferred embodiment, said facultative anaerobic bacteria are as defined and described above in the definitions and embodiments of facultative anaerobic bacteria. In another preferred embodiment, said aerotolerant anaerobic bacteria are as defined and described above in the definitions and embodiments of aerotolerant anaerobic bacteria. In a particular embodiment, the total amount of probiotics in said population of probiotics is any of those indicated above in the embodiments defining the amount of probiotics comprised in the biomaterial of the invention.

In some embodiments, the biomaterial of the invention is provided in a unit of biomaterial which has an area of about 1 m², 750 cm², 500 cm², 400 cm², 300 cm², 200 cm², 100 cm², 90 cm², 80 cm², 70 cm², 60 cm², 50 cm², 40 cm², 30 cm², 25 cm², 20 cm², 15 cm², 12 cm², 10 cm², 8 cm², 5 cm², 4 cm², 2 cm², 1 cm², 0.5 cm², preferably, of about 12 cm² In another particular embodiment, the thickness of said unit of biomaterial is of about 0.1 mm, 0.2 mm, 0.5 mm, 0.7 mm, 0.8 mm, 0.9 mm, 1 mm, 1.2 mm, 1.5 mm, 1.7 mm, 2 mm, 2.2 mm, 2.5 mm, 2.7 mm, 3 mm, 3.5 mm, 4 mm, 4.5 mm, 5 mm, 6 mm, 7 mm, 8 mm, 9 mm, 1 cm, 1.5 cm, 2 cm, 2.5 cm, 3 cm, 3.5 cm, 4 cm, 5 cm, 6 cm, 7 cm, 8 cm, 9 cm 10 cm, preferably of about 1.5 mm. In another particular embodiment, said unit of biomaterial of the invention is circular, rectangular, square, star-shaped, or with an irregular shape. In a preferred embodiment, it has a circular shape.

2—Methods for Preparing the Biomaterials According to the Invention

In a second aspect, the invention relates to a method for obtaining the biomaterial of the first aspect, comprising:
(i) culturing aerobic bacteria that produce cellulose simultaneously with facultative anaerobic probiotics and/or aerotolerant anaerobic probiotics under conditions suitable for the production of cellulose by the bacteria that produce cellulose, thereby resulting in a cellulose matrix containing the bacteria and the probiotics and,
(ii) incubating the cellulose matrix obtained in step (i) in a culture medium that provides conditions which are suitable for the proliferation of the probiotics in said matrix and which are not suitable the proliferation of the aerobic bacteria.

Said method is herein referred to as the method of the invention.

The expressions “aerobic bacteria”, “bacteria that produce cellulose”, “facultative anaerobic bacteria”, “aerotolerant anaerobic bacteria”, “probiotics”, “cellulose matrix” have been defined above, in connection with the biomaterial of the invention.

In a preferred embodiment, the aerobic bacteria are any of the aerobic bacteria specified in the aspect of the invention related to the biomaterial of the invention. In another preferred embodiment, the “probiotics” are any of the probiotics specified in the aspect related to the biomaterial of the invention. In another preferred embodiment, the facultative anaerobic bacteria are any of the facultative anaerobic bacteria specified in the aspect of the invention related to the biomaterial of the invention. In another particular embodiment, the “aerotolerant anaerobic bacteria” are any of the aerotolerant anaerobic bacteria specified in the aspect of the invention related to the biomaterial of the invention. In another preferred embodiment, “bacteria that produce cellulose”, are any of those specified in the aspect of the invention related to the biomaterial of the invention.

In a first step, the method of the invention comprises culturing aerobic bacteria that produce cellulose simultaneously with probiotics that are facultative anaerobic bacteria and/or aerotolerant anaerobic bacteria under conditions suitable for the production of cellulose by the bacteria that produce cellulose, thereby resulting in a cellulose matrix containing the bacteria and the probiotics and,

The expression “conditions suitable for the production of cellulose by the bacteria that produce cellulose” a used herein, refers to culture conditions that allow bacteria that produce cellulose, as defined in the aspect related to the biomaterial of the invention, to grow in a manner so that they produce bacterial cellulose.

In a particular embodiment, said culture conditions are aerobic culture conditions. In a preferred embodiment, aerobic culture conditions are achieved by simply performing the culture in an open atmosphere, i.e. in a flask not hermetically closed, or simply opened, in a culture room with an open atmosphere or even in the outdoor. The oxygen concentration in the atmosphere in which said culture is performed is of about 22%, 21%, 20.95%, 20.9%, 20.8%, 20.7%, 20.5%, 20.4%, 20.3%, 20.2%, 20.1%, 20%, 19.5%, 19%, 18%, 17%, 16%, 15%, 14%, 12%, 10%, preferably of about 21%, more preferably of about 20.95%.

In a particular embodiment, the culture medium of said culture conditions suitable for the production of cellulose by the bacteria that produce cellulose are commonly known by an expert in the field. Non-limiting examples of said mediums include Hestrin and Schramm (HS) medium, the composition of which is defined in Schramm M. and Hestrin S. 1954, J. Gen. Microbiol., 11 123-129, or in Costa A. S. et al. 2017, Frontiers in Microbiology, 8:2027, in particular in table 1 of said document. Other non-limiting examples of said mediums include variants of the HS medium, as defined in table 1 of Costa A. S. et al. 2017 supra. Additional non-limiting examples of medium suitable for the production of cellulose by the bacteria that produce cellulose are HS-ascorbic acid (HSA) medium, Hassid-Barker (HB) medium, Yamanaka medium, Zhou's medium, Son medium, Park medium, M1A05P5 medium, super optimal broth with catabolite, repression (SOC) medium, CSL-fructose (CSLFru), medium, fermentation medium (FM), yeast extract-peptone-dextrose (YPD) medium, acetate buffered medium (AB), modified (MHS) HS media, Joseph medium, fructose-corn steep solid solution (fru-CSS) medium and altered HS (AHS) medium, as defined in Hussain Z. et al. 2019, Cellulose, 26: 2895-2911, in particular in table 1 of said document, and any additional medium for the culture of bacteria that produce BC defined therein. Additional non-limiting examples of medium suitable for the production of cellulose by the bacteria that produce cellulose include fruit juice, sugar cane molasses, brewery waste and combinations thereof as defined in UI-Islam et al. 2017, Int. J. Biol. Macromol. 102, 1166-1173 and any additional media suitable for the culture of bacteria that produce BC defined therein.

In a preferred embodiment, the culture medium that allows the growth of the bacteria that produce cellulose is HS medium, or any variant thereof, preferably HS medium.

In a particular embodiment, step (i) of the method of the invention is performed in HS culture medium.

In some embodiments, the temperature of the culture conditions suitable for the production of cellulose by the bacteria that produce cellulose is between 15-50° C., 17° C.-45° C., 20° C.-40° C., 25° C.-37° C., 27° C.-35° C., 28° C.-32° C., 29° C.-31° C., preferably between 28° C.-32° C. In a particular embodiment, it is about 15° C., 17° C., 20° C., 21° C., 22° C., 23° C., 24° C., 25° C., 26° C., 27° C., 27.5° C., 28° C., 28.5° C., 29° C., 29.5° C., 30° C., 30.5° C., 31° C., 31.5° C., 32° C., 32.5° C., 33° C., 33.5° C., 34° C., 34.5° C., 35° C., 36° C., 37° C., 38° C., 39° C., 40° C., 42° C., 45° C., 47° C., 50° C., preferably about 30° C. Thus, in a particular embodiment, step (i) of the method of the invention is performed at about 30° C.

In a particular embodiment, culture conditions suitable for the production of cellulose by the bacteria that produce cellulose are static conditions or dynamic conditions. As understood by a skilled person, static conditions refer to culture conditions wherein the flask or container that contains the bacterial culture is static, i.e. not shaken nor stirred. Dynamic conditions refer to culture conditions wherein the culture medium and thus, preferably bacteria comprised in it as well, are in movement, preferably in a movement with a stable frequency.

In a particular embodiment, dynamic conditions are performed at about 10, 20, 30, 40 50, 55, 60, 65, 70, 75, 80, 85, 90, 96, 100, 105, 110, 115, 120, 125, 130, 135, 140, 145, 150, 155, 160, 165, 1701, 175, 180, 185, 190, 195, 200, 205, 210, 215, 220, 225, 230, 235, 240, 245, 250, 260, 270, 280, 290, 300, 310, 320, 350, 370, 400, 450, 500, 550, 600, 650, 700, 750, 750, 800, 850, 900, 1000 rpm, preferably at about 180 rpm. In another preferred embodiment, they are performed at about 200 rpm. In another particular embodiment, dynamic conditions are performed at 10-1000, 20-900, 30-800, 40-700, 50-600, 60-500, 70-450, 80-400, 90-350, 100-300, 110-250, 120-240, 130-230-140-220, 150-215, 160-210, 170-205, 180-200 rpm, preferably at 180-200 rpm.

Said conditions can be performed by methods well-known by an expert in the field, such as by carrying bacterial culture with a shaking flask, a stirring bioreactor, a flask put in an agitated platform or a rotator. In a particular embodiment, dynamic culture conditions are carried with a shaking flask or with a stirring bioreactor. In a preferred embodiment, dynamic culture conditions are carried with a shaking flask. In another preferred embodiment, dynamic culture conditions are carried with a stirring bioreactor. Thus, in a particular embodiment, step (i) of the method of the invention is performed under static conditions or dynamic conditions. In a preferred embodiment, step (i) of the method of the invention is performed under static conditions.

In another particular embodiment, the duration of the bacterial culture in the culture conditions suitable for the production of cellulose by the bacteria that produce cellulose is of about 12 h, 1 day, 1.5 days, 2 days, 2.5 days, 3 days, 3-5 days, 4 days, 4.5 days, 5 days, 5.5 days, 6 days, 6.5 days, 7 days, 7.5 days, 8 days, 8.5 days, 9 days, 9.5 days, 10 days, 1 days, 12 days, 15 days, 17 days, 20 days, 25 days, 30 days, 37 days, 45 days, 52 days, 60 days, preferably of about 1 day, even more preferably of about 2 days, yet more preferably of about 3 days. Thus, in a preferred embodiment, step (i) of the method of the invention is performed for about 1 day, even more preferably for about 2 days, yet more preferably for about 3 days.

In another particular embodiment, the duration of the of the bacterial culture in the culture conditions suitable for the production of cellulose by the bacteria that produce cellulose is of at least any of the time periods indicated in the embodiment above. Thus, in a particular embodiment, step (i) of the method of the invention is performed for at least 1 day, even more preferably, for at least 2 days, yet more preferably for at least 3 days.

The expression “culture simultaneously with”, as used herein, refers to the fact that the bacteria that produce BC and the probiotics are grown together forming part of the same culture. In embodiment, the culture is first inoculated with the bacteria that produce BC and, once the bacteria have reached sufficient concentration, the culture is inoculated with the probiotics and the culture is continued during the rest of step (i). In another embodiment, the culture is first inoculated with the probiotics and, once the probiotics have reached sufficient concentration, the culture is inoculated with the bacteria that produce BC and the culture is continued during the rest of step (i). In another embodiment, the culture is inoculated substantially at the same time with the probiotics and with the bacteria that produce BC and both types of cells are allowed to grow during the remaining of step (i) of the method of the invention.

In a particular embodiment, step (i) of the method of the invention is started by inoculation of a suspension of bacteria that produces BC and a suspension of probiotics in the culture medium. In a particular embodiment, the suspension of bacteria that produce BC inoculated in the culture medium to start step (i) is at an Optical Density at a wavelength of 600 nM (OD600) of about 0.1, 0.2, 0.3, 0.4, 0.5, 0.6, 0.7, 0.8, 0.9, 12, preferably about 0.3. In another particular embodiment, the suspension of probiotics inoculated in the culture medium to start step (i) is at an OD600 of about 0.1, 0.2, 0.3, 0.4, 0.5, 0.6, 0.7, 0.8, 0.9, 12, preferably about 0.4. In a particular embodiment, the volume of the suspension of bacteria that produce BC inoculated in the culture medium of step (i) is about 1%, 2%, 3%, 4%, 5%, 7%, 10%, 15%, 20%, 25%, 30%, 40%, 50% of the volume of culture medium of step (i), preferably about 10% of the volume of the culture medium of step (i). In another particular embodiment, the volume of the suspension of probiotics inoculated in the culture medium of step (i) is about 1%, 2%, 3%, 4%, 5%, 7%, 10% v/v, 15%, 20%, 25%, 30%, 40%, 50% of the volume of culture medium of step (i), preferably about 10% of the volume of culture medium of step (i)

Step (i) of the method of the invention is allowed to proceed until a cellulose matrix containing the bacteria and the probiotics is formed. In a preferred embodiment, a cellulose matrix containing the bacteria and the probiotics is considered to be formed when a cellulose matrix appears in the culture medium showing a thickness of at least 50 μm, 75 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 325 μm, 350 μm, 375 μm, 400 μm, 425 μm, 450 μm, 475 μm, 500 μm, 600 μm, 700 μm, 800 μm, 900 μm, 1 mm, 1.5 mm, 2 mm, 2.5 mm, 3 mm, 3.5 mm, 4 mm, 4.5 mm, 5 mm, 6 mm, 7 mm, 8 mm, 9 mm, 1c m, preferably of at least 200 μm. Methods for identifying BC, or a cellulose matrix in a culture of bacteria that produce cellulose, are provided in the definition of BC in the aspect of the invention related to the biomaterial of the invention.

In a second step, the method of the invention comprises incubating the cellulose matrix obtained in step (i) in a culture medium that provides conditions which are suitable for the proliferation of the probiotics in said matrix and which are not suitable the proliferation of the aerobic bacteria.

In a particular embodiment, the second step of the method of the invention is performed by substantially removing the culture medium with which step (i) is performed from the container in which step (i) is performed, and adding to the container where the cellulose matrix obtained from step (i) is comprised, the culture medium with which step (ii) is performed. In one embodiment, the cellulose matrix can be washed for one or more times in order to remove rests of any component found in the medium used in step (i) before adding the culture medium with which step (ii) is performed to the container where the cellulose matrix obtained from step (i) is comprised.

In a preferred embodiment, the container in which step (i) is performed is the same container in which the culture medium with which step (ii) is performed is added. In another embodiment, the container in which step (i) is performed is different from the container in which the culture medium with which step (ii) is performed is added.

The expression “substantially removing the culture medium with which step (i) is carried from the container under which step (i) is carried”, as used herein, refers to removing about 100%, 99.9%, 99.8%, 99.7%, 99.6%, 99.5%, 99.4%, 99.4%, 99.3%, 99.2%, 99.1%, 99%, 98%, 97%, 96%, 95%, 94%, 93%, 92%, 91%, 90%, 89%, 87%, 85%, 82%, 80%, 75%, 72%, 70%, 65%, 60%, 50%, of the culture medium with which step (i) is carried from the container in which step (i) is carried, preferably about 100% of the culture medium with which step (i) is carried from the container in which step (i) is carried. Methods allowing to determine the amount of culture medium removed from a container are well known by an expert in the field, and can simply consist on determining the volume of the culture medium comprised in said container just before removing any culture medium (such as by transferring the culture medium to a flask comprising marks indicating different volume amounts), and the volume of culture medium removed from said container (such as a by transferring the culture medium removed to such a flask comprising marks indicating different volume amounts).

In a particular embodiment, step (ii) of the method of the invention is performed by recovering the matrix from the culture of step (i) and transferring it to a second culture vessel containing the appropriate culture medium for step (ii). In one embodiment, the cellulose matrix recovered can be washed for one or more times in order to remove rests of the any component found in the medium used in step (i).

In a particular embodiment, the expression “conditions which are suitable for the proliferation of the probiotics in said matrix and which are not suitable the proliferation of the aerobic bacteria”, as used herein, refer to culture conditions that allow probiotics to grow, but not bacteria aerobic bacteria, preferably those referred in step (i) of the method.

In a particular embodiment, said conditions of the culture medium of step (ii) of the method are anaerobic conditions. Thus, in a preferred embodiment, step (ii) of the method of the invention is performed by incubating the cellulose obtained in step (i) in a culture medium under an anaerobic atmosphere. In a preferred embodiment, anaerobic conditions are performed by placing the flask in which said bacteria are cultured in a culture room, or box with a controlled atmosphere. In another particular embodiment, the flask comprising the bacterial culture is hermetically sealed and the atmosphere within the flask is controlled. In a particular embodiment, the controlled atmosphere referred in the two previous embodiments is characterized by an oxygen concentration below 21%, 20%, 18%, 17%, 15%, 12%, 10%, 8%, 7%, 6%, 5%, 4%, 3%, 2%, 1%, 0.9%, 0.75%, 0.5%, 0.25%, 0.1%, 0.09%. 0.08%, 0.05%, 0.025%, 0.01%, preferably below 8%.

In another particular embodiment, the culture medium of step (ii) of the method of the invention is selected form the group consisting of Man, Rogosa, and Sharpe (MRS) medium, Reinforced Clostridial Medium (RCM), M17, Brain Heart Infusion (BHI), HANK'S medium, APT medium, LM17 medium, GM17 medium, Elliker medium, Tryptone Phytone Yeast (TPY), glucose blood liver (BL), Columbia (CLB), Liver cysteine lactose (LCL), modified MRS (mMRS), modified MRS and blood (mMRS+blood), modified BL with blood (mBL), modified RCM (mRCM), RCPB and the like. A description of MRS medium and other media appropriate for the culture of probiotics can be found in Handbook of Culture Media for Food Microbiology, Vol. 34, edited by Janet E. L. Corry, G. D. W. Curtis, Rosamund M. Baird.

In some embodiments, when the probiotics comprise bacteria from the genus Lactobacillus, the culture medium to be used in step (ii) of the method of the invention is Man, Rogosa, and Sharpe (MRS) medium, Reinforced Clostridial Medium (RCM), M17, Brain Heart Infusion (BHI), HANK'S medium, APT medium, LM17 medium, GM17 medium or Elliker medium. In other embodiments, when the probiotics comprise bacteria from the genus Bifidobacterium, the culture media to be used in step (ii) of the method includes MRS, Tryptone Phytone Yeast (TPY), glucose blood liver (BL), Columbia (CLB), Liver cysteine lactose (LCL), modified MRS (mMRS), modified MRS and blood (mMRS+blood), modified BL with blood (mBL), modified RCM (mRCM), RCPB and the like.

In a preferred embodiment, the culture medium in which step (ii) of the method of the invention is performed is MRS medium.

In some embodiments, the temperature of the culture conditions that are suitable for the proliferation of the probiotics in the BC matrix and which are not suitable the proliferation of the aerobic bacteria is between 15-60° C., 17° C.-50° C., 20° C.-47° C., 25° C.-45° C., 30° C.-40° C., 35° C.-39° C., 36° C.-38° C., more preferably, between 36° C.-38° C. In a more preferred embodiment, said culture conditions are performed at a temperature of about 15° C., 17° C., 20° C., 21° C., 22° C., 23° C., 24° C., 25° C., 26° C., 27° C., 27.5° C., 28° C., 28.5° C., 29° C., 29.5° C., 30° C., 30.5° C., 31° C., 31.5° C., 32° C., 32.5° C., 33° C., 33.5° C., 34° C., 34.5° C., 35° C., 36° C., 37° C., 38° C., 39° C., 40° C., 42° C., 45° C., 47° C., 50° C., 55° C., 60° C., preferably at about 37° C. Thus, in a preferred embodiment, step (ii) of the method of the invention is performed at about 37° C.

In some embodiments, culture conditions that are suitable for the proliferation of the probiotics in the BC matrix and which are not suitable the proliferation of the aerobic bacteria are static conditions, or dynamic conditions. Static and dynamic culture conditions have been defined above in connection with the culture conditions of the method of the invention suitable for the production of cellulose by the bacteria that produce cellulose. Definitions and embodiments addressed to said static and dynamic culture conditions apply to the static and dynamic conditions of the culture conditions suitable for the proliferation of the probiotics in the BC matrix and which are not suitable the proliferation of the aerobic bacteria. In a preferred embodiment, step (ii) of the method of the invention is performed under static conditions or dynamic conditions. In a preferred embodiment, step (ii) of the method of the invention is performed under static conditions.

In some embodiments, step (ii) of the method of the invention is carried out for about 12 h, 1 day, 1.5 days, 2 days, 2.5 days, 3 days, 3-5 days, 4 days, 4.5 days, 5 days, 5.5 days, 6 days, 6.5 days, 7 days, 7.5 days, 8 days, 8.5 days, 9 days, 9.5 days, 10 days, 1 days, 12 days, 15 days, 17 days, 20 days, 25 days, 30 days, 37 days, 45 days, 52 days, 60 days, preferably for about 1 day, even more preferably for about 2 days.

In particular embodiment, culture conditions that are suitable for the proliferation of the probiotics in the BC matrix and which are not suitable the proliferation of the aerobic bacteria are performed for a period of time of at least any of those indicated in the previous embodiment. Thus, in a preferred embodiment, step (ii) of the method of the invention is performed for at least 1 day, even more preferably for at least 2 days.

In a particular embodiment, the culture medium is renewed after about 6 h, 12 h, 15 h, 1 day, 2 days, 2.5 days, 3 days, 3-5 days, 4 days, 4.5 days, 5 days, 5.5 days, 6 days, 6.5 days, 7 days, 7.5 days, 8 days, 8.5 days, 9 days, 9.5 days, 10 days, preferably after about 12 hours, more preferably after about 1 day.

As understood by a skilled person, culture conditions that are suitable for the proliferation of the probiotics in the BC matrix and which are not suitable for the proliferation of the aerobic bacteria are applied to the aerobic bacteria and probiotics comprised in the BC matrix obtained at the end of step (i).

In a particular embodiment, the BC obtained at the end of step (i) is rinsed before carrying step (ii) of the method of the invention. Thus, in a preferred embodiment, an additional step of rinsing the cellulose is carried out between step (i) and (ii). In a particular embodiment, the cellulose is rinsed with water. In another particular embodiment, the cellulose is rinsed with the same culture medium that is to be used in step (ii), such as one of the culture medium provided above for the culture conditions that are suitable for the proliferation of the probiotics in the BC matrix and which are not suitable the proliferation of the aerobic bacteria. In some embodiments, BC obtained at the end of step (i) is rinsed 1, 2, 3, 4, 5, 6, 7, 8, 9, 10 times, preferably 1 time before carrying step (ii) of the method of the invention.

In a particular embodiment, the aerobic bacteria that produce cellulose are the aerobic bacteria that produce the BC described in the aspect of the invention related to the biomaterial of the invention. Thus, the aerobic bacteria that produce cellulose are any of the aerobic bacteria that produce BC specified in said aspect of the invention.

In a preferred embodiment, the aerobic bacteria which are used in the method of the invention are from the genus Acetobacter, Gluconacetobacter Komagataeibacter, or combinations thereof.

In another preferred embodiment, the aerobic bacteria from the genus Acetobacter are form the species A. xylinum, A. nitrogenifigens, A. orientalis or combinations thereof, preferably form the species A. xylinum. More preferably the aerobic bacteria from the genus Acetobacter are from the strain deposited at the Colección Española de Cultivos Tipo (CECT) with accession number CECT 473.

In another preferred embodiment, the aerobic bacteria from the genus Gluconacetobacter are from the species G. hansenii, G. swingsii, G. sacchari, G. kombuchae, G. entanii, G. persimmonis, G. sucrofermentans or combinations thereof.

In another preferred embodiment, the aerobic bacteria from the genus Komagataeibacter are from the species K. europaeus, K. medellinensis, K. intermedius, K. rhaeticus, K. kakiaceti, K. oboediens, K. nataicola, K. saccharivorans, K. maltaceti or combinations thereof.

In another particular embodiment, the probiotics of the method of the invention are as those described in the aspect related to the biomaterial of the invention. Thus, they are any of the probiotics specified in said aspect of the invention.

In a particular embodiment, the probiotics of the method of the invention are facultative anaerobic bacteria. In another particular embodiment, the probiotics of the method of the invention are aerotolerant anaerobic bacteria. In another particular embodiment, the probiotics of the method of the invention are facultative anaerobic bacteria or aerotolerant anaerobic bacteria. In a preferred embodiment, the probiotics of the method of the invention are facultative anaerobic bacteria and/or aerotolerant anaerobic bacteria.

In a particular embodiment, the probiotics of the method of the invention as described herein are from the genus Lactobacillus, Bifidobacterium, Lactococcus, Streptococcus, Enterococcus, Pediococcus, Leuconostoc, Bacillus, Escherichia or combinations thereof. In a preferred embodiment, the probiotics of the method of the invention as described herein are from the genus Lactobacillus, Bifidobacterium, Lactococcus, Streptococcus, or combinations thereof. In another preferred embodiment, they are from the genus Lactobacillus. In an embodiment, they are from the genus Bifidobacterium.

In a particular embodiment, probiotics of the method of the invention that are from the genus Lactobacillus are from the species L. fermentum, L. gasseri, L. acidophilus, L. plantarum., L. rhamnosus, L. casei, L. johnsonii, L. delbrueckii, L. salivarus, or combinations thereof. In a preferred embodiment, they are form the species L. fermemtum. In another preferred embodiment, the probiotics of the method of the invention are from the species L. gasseri.

In a preferred embodiment, probiotics of the method of the invention are from the genus Lactobacillus, preferably from the species L. fermentum, L. gasseri, L. acidophilus, L. plantarum., L. rhamnosus, L. casei, L. johnsonii, L. delbrueckii, L. salivarus, or combinations thereof. In a preferred embodiment, they are form the species L. fermemtum. In another preferred embodiment, the probiotics of the method of the invention are from the species L. gasseri.

In a particular embodiment, the probiotics of the method of the invention that are from the species L. acidophilus are from the strain CECT 903.

In another particular embodiment, the probiotics of the method of the invention that are from the species L. plantarum are from the strain CECT 220.

In another particular embodiment, the probiotics of the method of the invention that are from the species L. rhamnosus are from the strain CECT 278.

In a particular embodiment, the probiotics of the method of the invention that are from the species Bifidobacterium are from the species B. breve, B. longum, B. animalis, B. infantum, B. animalis, lactis, Bifidobacterium thermophilum, Bifidobacterium boum, Bifidobacterium minimum, Bifidobacterium pyschraerophilum or combinations thereof. In another particular embodiment, the probiotics of the method of the invention that are from the species Bifidobacterium are from the species B. breve, B. longum, B. animalis, B. infantum, B. animalis, or combinations thereof. In a preferred embodiment, they are from the species B. breve. In a preferred embodiment, the probiotics of the method of the invention are from the species B. breve.

In a particular embodiment, the probiotics of the method of the invention are from the species Bifidobacterium, preferably from the species B. breve, B. longum, B. animalis, B. infantum, B. animalis, lactis, Bifidobacterium thermophilum, Bifidobacterium boum, Bifidobacterium minimum, Bifidobacterium pyschraerophilum or combinations thereof. In another embodiment, the probiotics of the method of the invention are from the species Bifidobacterium, preferably from the species B. breve, B. longum, B. animalis, B. infantum, B. animalis, or combinations thereof. In a preferred embodiment, they are from the species B. breve. In a preferred embodiment, the probiotics of the method of the invention are from the species B. breve.

In a particular embodiment, the probiotics of the method of the invention that are from the species Lactococcus are from the species L. lactis.

In a particular embodiment, the probiotics of the method of the invention that are from the species Streptococcus are from the species S. thermophiles.

In a preferred embodiments, the probiotics of the method of the invention that are facultative anaerobic bacteria are any of the facultative anaerobic bacteria specified in the aspect of the invention related with the biomaterial of the invention. Thus, all the embodiments addressed to the probiotics comprised in the biomaterial of the invention that are facultative anaerobic bacteria, apply to the probiotics of the biomaterial of the invention that are facultative anaerobic bacteria.

In a particular embodiment, probiotics of the method of the invention that are facultative anaerobic bacteria are from the genus Lactobacillus, Bifidobacterium, Lactococcus, Streptococcus, Enterococcus, Pediococcus, Leuconostoc, Bacillus, Escherichia or combinations thereof. In another particular embodiment, the probiotics of the method of the invention that are anaerobic facultative bacteria are from the genus Lactobacillus, Bifidobacterium, Lactococcus, Streptococcus, or combinations thereof. In another embodiment, probiotics of the method of the invention that are anaerobic facultative bacteria are from the genus Lactobacillus, Lactococcus, Streptococcus, Enterococcus, Pediococcus, Leuconostoc, Bacillus, Escherichia or combinations thereof. In another embodiment, probiotics of the method of the invention that are anaerobic facultative bacteria are from the genus Lactobacillus, Lactococcus, Streptococcus, or combinations thereof.

In a particular embodiment, the probiotics of the method of the invention that are facultative anaerobic bacteria that are from the genus Lactobacillus are from the species L. fermentum, L. gasseri, L. acidophilus, L. plantarum, L. rhamnosus, L. casei, L. johnsonii, L. delbrueckii, L. salivarus, or combinations thereof. In preferred embodiment, the probiotics of the method of the invention that are facultative anaerobic bacteria are form the species L. fermemtum. In another preferred embodiment, probiotics of the method of the invention that are facultative anaerobic bacteria are from the species L. gasseri.

In a preferred embodiment, probiotics of the method of the invention are facultative anaerobic bacteria from the genus Lactobacillus, preferably from the species L. fermentum, L. gasseri, L. acidophilus, L. plantarum, L. rhamnosus, L. casei, L. johnsonii, L. delbrueckii, L. salivarus, or combinations thereof. In preferred embodiment, probiotics of the method of the invention are facultative anaerobic bacteria from the species L. fermemtum. In another preferred embodiment, probiotics of the method of the invention are facultative anaerobic bacteria from the species L. gasseri.

In a preferred embodiment, the probiotics of the method of the invention that are aerotolerant anaerobic bacteria are any of the aerotolerant anaerobic bacteria specified in the aspect of the invention related with the biomaterial of the invention. Thus, all the embodiments addressed to the probiotics comprised in the biomaterial of the invention that are aerotolerant anaerobic bacteria, apply to the probiotics of the biomaterial of the invention that are aerotolerant anaerobic bacteria.

In a particular embodiment, the probiotics of the method of the invention that are aerotolerant anaerobes are from the genus Bifidobacterium. In a particular embodiment, the probiotics of the method of the invention that are aerotolerant anaerobes that are from the genus Bifidobacterium are from the species B. breve, B. longum, B. animalis, B. infantum, B. animalis, lactis, Bifidobacterium thermophilum, Bifidobacterium boum, Bifidobacterium minimum, Bifidobacterium pyschraerophilum or combinations thereof. In another particular embodiment, the probiotics of the method of the invention that are aerotolerant anaerobes are from the genus Bifidobacterium animalis subsp. lactis, Bifidobacterium thermophilum, Bifidobacterium boum, Bifidobacterium minimum, Bifidobacterium pyschraerophilum, and combinations thereof. In another particular embodiment, the probiotics of the method of the invention that are aerotolerant anaerobes are from the genus Bifidobacterium, preferably from the species B. breve, B. longum, B. animalis, B. infantum, B. animalis or combinations thereof. In a preferred embodiment, the probiotics of the method of the invention that are aerotolerant anaerobes are from the species B. breve.

In a particular embodiment, the probiotics of the method of the invention are aerotolerant anaerobes from the genus Bifidobacterium, preferably from the species B. breve, B. longum, B. animalis, B. infantum, B. animalis, lactis, Bifidobacterium thermophilum, Bifidobacterium boum, Bifidobacterium minimum, Bifidobacterium pyschraerophilum or combinations thereof. In another particular embodiment, probiotics of the method of the invention are aerotolerant anaerobes from the species Bifidobacterium animalis subsp. lactis, Bifidobacterium thermophilum, Bifidobacterium boum, Bifidobacterium minimum, Bifidobacterium pyschraerophilum, and combinations thereof. In another particular embodiment, of the method of the invention are aerotolerant anaerobes from the genus Bifidobacterium, preferably from the species B. breve, B. longum, B. animalis, B. infantum, B. animalis or combinations thereof. In a preferred embodiment, of the method of the invention are aerotolerant anaerobes from the species B. breve.

In a particular embodiment, the probiotics of the method of the invention comprise bacteria form the genus Bifidobacterium, preferably from the species B. breve, and the culture medium of step (i) of the method is enriched with cysteine. In a particular embodiment, said culture medium is enriched with about 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 15 μg/ml, 20 μg/ml of cysteine, preferably with about 5 μg/ml of cysteine. In another particular embodiment, said medium is HS medium. Thus, in a preferred embodiment, the probiotics of the method of the invention comprise bacteria form the genus Bifidobacterium, preferably from the species B. breve, and step (i) is performed in HS culture medium enriched with cysteine, preferably with about 5 μg/ml of cysteine.

In another particular embodiment, the probiotics of the method of the invention comprise bacteria from the genus Bifidobacterium, preferably from the species B. breve, and the culture medium of step (ii) of the method is enriched in cysteine. In a particular embodiment, said culture medium is enriched with about 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 15 μg/ml, 20 μg/ml of cysteine, preferably with about 5 μg/ml of cysteine. In another particular embodiment, said medium is MRS medium. Thus, in a preferred embodiment, the probiotics of the method of the invention comprise bacteria from the genus Bifidobacterium, preferably from the species B. breve, and the culture media is MRS medium enriched in cysteine, preferably with about 5 μg/ml of cysteine.

In a particular embodiment, step (ii) of the method of the invention is allowed to proceed until the amount of bacteria that produce BC comprised in a weight unit of cellulose matrix (or BC) is bellow a reference value. In a preferred embodiment, said reference value is any of the % of bacteria that produce BC indicated in the definition of “essentially free” in the aspect of the invention related with the biomaterial of the invention. Thus, in a preferred embodiment, the reference value is 15%, 12%, 10%, 9%, 7%, 5%, 3%, 2%, 1.7%, 1.5%, 1.4%, 1.3%, 1.2%, 1.1%, 1%, 0.9%, 0.8%, 0.7%, 0.6% 0.5%, 0.4%, 0.3%, 0.2%, 0.1%, 0.09%, 0.085%, 0.08%, 0.07%, 0.06%, 0.005%, 0.04%, 0.03%, 0.02%, 0.01%, 0.009%, 0.008%, 0.007%, 0.006%, 0.005%, 0.004%, 0.003%, 0.002%, 0.001% of cellulose producing bacteria with respect to the amount of probiotics per weight unit of BC.

In another particular embodiment, step (ii) of the method of the invention is allowed to proceed until the amount of probiotics comprised in a weight unit of cellulose matrix (or BC) is above a reference value. In a preferred embodiment, said reference value is any of the amount of probiotic bacteria (in CFU of probiotic bacteria per mg of BC) indicated in any of the embodiments addressed to the amount of probiotics comprised in the biomaterial of the invention in the aspect of the invention addressed the biomaterial of the invention. Thus, in a preferred embodiment, the reference value is 8.7×10¹⁰ CFU of probiotic bacteria per mg BC, preferably 9.2×10¹⁰ CFU of probiotic bacteria per mg of BC, more preferably 1×10¹¹ CFU of probiotic bacteria per mg of BC, yet more preferably 1.2×10¹¹ CFU of probiotic bacteria per mg of BC, even yet more preferably 1.4×10¹¹ CFU of probiotic bacteria per mg of BC, even more preferably 1.7×10¹¹ CFU of probiotic bacteria per mg of BC. In a preferred embodiment, the reference value is 1.2×10¹¹ CFU of probiotic bacteria per mg of BC. In another preferred embodiment, the reference value is 1×10¹¹ CFU of probiotic bacteria per mg of BC.

Methods allowing determining the amount of probiotics in a weigh unit of BC and of bacteria that produce BC with respect to the amount of probiotics in the biomaterial (for instance, with respect to the amount of probiotics per weight unit of BC) are described in the aspect of the invention related with the biomaterial of the invention.

In another embodiment, step (ii) of the method of the invention is allowed to proceed for any of the time periods indicated in any of the embodiments above. In a particular embodiment, the definitions and embodiments of the previous aspect of the invention apply to the method of the invention.

3. Biomaterial Obtained by the Method of the Invention.

A third aspect of the invention relates to a biomaterial obtained or obtainable by the method of the second aspect of the invention.

Said biomaterial is referred to as the biomaterial obtained by the method of the invention.

In a particular embodiment, said biomaterial is as the biomaterial of the invention. Thus, all the definitions, descriptions and embodiments of the aspect of the invention related with the biomaterial of the invention apply to the biomaterial obtained by the method of the invention. As understood by a skilled person, said method is as defined and described in the aspect of the invention related with the method of the invention.

In a particular embodiment, the definitions and embodiments provided in any of the aspects above apply to the biomaterial obtained by the method of the invention.

4. Medical Uses and Pharmaceutical Compositions of the Invention

The authors of the present invention have observed that the biomaterial of the invention containing BC and probiotics shows antibacterial activity against bacteria that are common pathogens and cause infectious diseases (in particular against S. aureus and P. aeruginosa). Additionally, since the biomaterial of the invention is a solid biomaterial, it can be advantageously used for the preparation of films and patches for the treatment of different diseases (see example 4).

Thus, a fourth aspect of the invention relates to the biomaterial of the first or third aspect of the invention, for use in medicine.

A fifth aspect of the invention relates to the biomaterial of the first or third aspect of the invention, for use in the treatment of a wound or of a bacterial infection.

Said medical uses are herein referred to as the medical uses of the invention.

A sixth aspect of the invention refers to a pharmaceutical composition comprising the biomaterial of the first or third aspect of the invention, and a pharmaceutically acceptable carrier.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or condition, or the disease or health condition progression. For example, a disclosed method for reducing the effects of a bacterial infection is considered to be a treatment if there is a 10% reduction in one or more symptoms of the infection in a subject with the infection when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition (e.g. bacterial vaginosis, impetigo, bacterial cellulitis, mastitits, etc).

The term “wound”, as used herein refers to an injury to a living tissue caused by a cut, blow, or other impact, wherein the skin is typically cut or broken. In a particular embodiment, the wound is infected, preferably by bacteria. In another particular embodiment, the bacterial infection of said wound is as the bacterial infections described below. As understood by a skilled person, the existence of a wound or of an infected wound is considered a health condition as referred in the definition of “treatment”.

The term “infection”, as used herein, refers to a condition characterized by the invasion of an organism's tissue of a subject (the host), preferably a human, by a disease-causing, or pathogenic, microorganism, its growth and multiplication. It generally involves a reaction of the host organism to try to stop the growth and multiplication of the pathogenic organism. Said reaction might be characterized by erythema, edema, warmth, and pain or tenderness. Another common symptom of infection is fever (i.e. a body temperature higher than a reference level, wherein the reference level is 37° C. in humans). The affected area may also become dysfunctional (eg, hands and legs) depending on the severity of the infection. Pathogenic microorganisms include bacteria, virus, fungi and parasites. In a particular embodiment, the term infection refers to an infection caused by bacteria, or to a bacterial infection. As understood by a skilled person, the existence of a wound or of an infected wound is considered a health condition, and in some cases a disease, as referred in the definition of “treatment”.

The term “bacterial infection”, as used herein, refers to an infection in a living tissue of a subject, preferably of a human, wherein the microorganisms that cause the infection are bacteria. Non-limiting examples of bacteria that cause bacterial infections are bacteria from the genus Staphylococus, Pseudomonas, Streptococcus, Salmonella, Neisseria, Brucella, Mycobacterium, Nocardia, Listeria, Francisella, Legionella, and bacteria from the species Pseudomonas aeruginosa, Burkholderia cenocepacia, Mycobacterium avium, Mycobacterium tuberculosis, Escherichia colli, Yersinia pestis, or combinations thereof. Thus, in a particular embodiment, bacterial infection referred in the medical uses of the invention are caused by any of the just mentioned bacteria. In another particular embodiment, bacterial infections referred in the medical uses of the invention are caused by a combination of bacteria form those just mentioned.

In a particular embodiment, bacterial infections referred in the present invention are caused by bacteria selected from the group consisting of bacteria from the genus Staphylococus, bacteria from the genus Pseudomonas, and a combination of bacteria form the genus Staphylococus and from the genus Pseudomonas. In a preferred embodiment, the bacteria from the genus Staphylococcus are from the species Staphylococcus aureus. In another preferred embodiment, the bacteria from the genus Pseudomonas are from the species Pseudomonas aeruginosa. In another preferred embodiment, bacterial infections referred in the present invention are caused by a combination of Staphylococcus aureus and of Pseudomonas aeruginosa.

In a particular embodiment, the infection referred in the medical uses of the invention is caused by Staphylococcus aureus or Pseudomonas aeruginosa.

In a particular embodiment, the infection is a topical infection. The term “topical infection”, as used herein refers to an infection of a surface of the body. It thus refers to infections of the skin, or of any mucosa. The term “mucous membrane” or “mucosa”, as used herein, includes a membrane that lines various cavities in the body and covers the surface of internal organs. It consists of one or more layers of epithelial cells overlying a layer of loose connective tissue. It is mostly of endodermal origin and is continuous with the skin at various body openings such as the eyes, ears, inside the nose, inside the mouth, lip, vagina, the urethral opening and the anus. Non-limiting examples of mucosa include bronchial mucosa and the lining of vocal folds, endometrium, esophageal mucosa, gastric mucosa, intestinal mucosa, nasal mucosa, olfactory mucosa, oral mucosa, penile mucosa, vaginal mucosa, frenulum of tongue, tongue, anal canal, palpebral conjunctiva, urinary tract mucosa, bladder mucosa.

Non-limiting examples of topical infection include infections of the skin, mastitis, otitis, ecthyma, erythema, erysipelas, bacterial cellulitis, folicullitis, furunculosis, hydrosadenitis, paronychia, infection in atopic dermatitis, superinfection in atopic dermatitis, conjunctivitis, staphylococcal blepharitis, wound infection, a burn infection, bacterial vaginosis, infection from the urinary tract, infection of cardiac valves, or in some case, infection of a joint. In a preferred embodiment, said infections are bacterial infections.

In another particular embodiment, the infection is an infection of a soft tissue. The term “soft tissue”, as used herein, refers to tissues that connect, support, or surround other structures and organs of the body, not being hard tissue such as a bone. Soft tissue includes tendons, ligaments, fascia, skin, fibrous tissues, fat, synovial membranes (which are connective tissue), muscles, nerves and blood vessels (which are not connective tissue). Non-limiting examples of infections of a soft tissue include any of those referred in the definition of topical infection, and in particular, bacterial vaginosis, infection from the urinary tract, infection of cardiac valves, or in some case, infection of a joint. In a preferred embodiment, said infections are bacterial infections.

In another particular embodiment, the bacterial infection is an infection of a non-soft tissue, such as a bone, a part of the joint that is not a soft tissue (such as cartilage or synovial fluid), or a prosthesis. Thus, non-limiting examples of such infections include infection of artificial cardiac valves, bone infection, or joint infection. In a preferred embodiment, said infections are bacterial infections.

In another preferred embodiment, the bacterial infection referred in the medical uses of the invention is selected from the group consisting of bacterial vaginosis, mastitis, and otitis, impetigo, ecthyma, erythema, erysipelas, bacterial cellulitis, folicullitis, furunculosis, hydrosadenitis, paronychia, infection in atopic dermatitis, superinfection in atopic dermatitis, ocular infection, infection from the urinary tract, infection of cardiac valves, infection of artificial cardiac valves, bone infection, joint infection, wound infection, and a burn infection.

The term “bacterial vaginosis”, “BV”, or “vaginosis”, as used herein refers to a health condition considered as the most frequent cause of vaginal disorders in women of reproductive age. The most common symptoms of this infection are the dense abnormal vaginal discharge, pain, itching and an unpleasant odor. BV is characterized by the alteration of the normal balance of the vaginal microbiota, and the pathological condition typically referred to as ‘vaginal dysbiosis’. The native microbiota helps to create a protective barrier for vaginal mucosa against infections. However, the healthy microbiota is sensitive to several factors, in particular, to changes in the pH of the mucosa, which can unbalance the microbial populations, favoring the appearance of infection-causing microbes. The health of the vaginal microbiota is believed to be maintained by lactic acid-producing organisms, such as Lactobacilli. It has been reported that: S. aureus is the most prevalent cause of BV, followed by E. Coli. P. aeruginosa has also been identified as a cause of BV.

The term “mastitis”, as used herein, refers to a health condition characterized by an inflammation of the breast or udder, usually associated with breastfeeding. Symptoms typically include local pain and redness. There is often an associated fever and general soreness. Onset is typically fairly rapid and usually occurs within the first few months of delivery. Risk factors include poor latch, cracked nipples, use of a breast pump, and weaning. Complications can include abscess formation. The bacteria most commonly involved are Staphylococcus and Streptococci, in particular Staphylococcus aureus. Diagnosis is typically based on symptoms. Ultrasound may be useful for detecting a potential abscess.

The term “otitis” as used herein refers to a health condition characterized by the inflammation of the ear, generally caused by a bacterial infection. It can be subdivided in otitis externa, otitis media and otitis interna or labyrinthitis. The term “otitis externa”, “external otitis”, or “swimmer's ear”, refers to an otitis that involves the outer ear and ear canal. In external otitis, the ear hurts when touched or pulled. The term “otitis media”, or “middle ear infection”, refers to an otitis that involves the middle ear. In otitis media, the ear is infected or clogged with fluid behind the ear drum, in the normally air-filled middle-ear space. This very common childhood infection sometimes requires a surgical procedure called myringotomy and tube insertion. The term “otitis interna”, or “labyrinthitis”, refers to an otitis that involves the inner ear. The inner ear includes sensory organs for balance and hearing. When the inner ear is inflamed, vertigo is a common symptom. It has been reported that the most prevalent cause of otitis is S. aureus or P. aeruginosa.

The term “impetigo”, as used herein, refers to a health condition characterized by bacterial infection that involves the superficial skin. The most common presentation is yellowish crusts on the face, arms, or legs. Less commonly there may be large blisters which affect the groin or armpits. The lesions may be painful or itchy. Fever is uncommon. It is typically due to either Staphylococcus aureus or Streptococcus pyogenes.

The term “ecthyma”, as used herein, refers to refers to a health condition which is a variation of impetigo, presenting at a deeper erosion of the skin, such as erosions into the dermis. It is well known to be caused by group A beta-hemolytic streptococci (such as Streptococcus pyogenes or Streptococcus dysgalactiae). Concomitant Staphylococcus aureus is often isolated from lesional skin. On occasion, S. aureus alone has been isolated. It is often referred to as a deeper form of impetigo.

The term “erythema”, as used herein, refers to a health condition characterized by redness of the skin or mucous membranes, caused by hyperemia (increased blood flow) in superficial capillaries. It occurs with any skin injury, infection, or inflammation. It ca be caused by infection, which can cause the capillaries to dilate, resulting in redness. Erythema disappears on finger pressure (blanching). It can be caused by bacteria from the genus Staphylococus bacteria, in particular by S. aureus.

The term “erysipelas”, as used herein, refers to a health condition characterized by a bacterial infection of the upper dermis extending to the subcutaneous lymphatic vessels which causes a rash characterized by a well-defined area or areas of bright red, inflamed and rough or leathery skin. It usually affects skin on the face, arms, legs, hands and feet. It is generally caused by beta-hemolytic group A Streptococcus bacteria (such as streptococuspyogenes, or streptococcus dysgalactiae) on scratches or otherwise infected areas. It can also be caused by Staphylococcus aureus. Erysipelas is more superficial than cellulitis, and is typically more raised and demarcated.

The expression “bacterial cellulitis” or “cellulitis”, as used herein, refers to a health condition characterized by a bacterial infection involving the inner layers of the skin. It specifically affects the dermis and subcutaneous fat. Signs and symptoms include an area of redness which increases in cm over a few days. The borders of the area of redness are generally not sharp and the skin may be swollen. While the redness often turns white when pressure is applied, this is not always the case. The area of infection is usually painful. Lymphatic vessels may occasionally be involved, and the person may have a fever and feel tired. The legs and face are the most common sites involved, though cellulitis can occur on any part of the body. The leg is typically affected following a break in the skin. Other risk factors include obesity, leg swelling, and old age. For facial infections, a break in the skin beforehand is not usually the case. The bacteria most commonly involved are Streptococcus and Staphylococcus aureus.

The term “folicullitis”, as used herein, refers to a health condition characterized by the infection and inflammation of one or more hair follicles. The condition may occur anywhere on the skin except the palms of the hands and soles of the feet. Generally it is caused by bacteria from the species Staphylococcus aureus.

The term “furunculosis” or “carbuncle”, as used herein, refers to a health condition characterized by a cluster of boils typically filled with purulent exudate (dead neutrophils, phagocytized bacteria, and other cellular components), caused by bacterial infection, most commonly with Staphylococcus aureus or Streptococcus pyogenes. Carbuncles may develop anywhere, but they are most common on the back and the nape of the neck.

The term “hidrosadenitis”, “hidrosadenities supurativa”, “hidradenitis”, “hidradenitis supurativa”, or “acne inversa” is a long-term skin disease characterized by the occurrence of inflamed and swollen lumps. These are typically painful and break open, releasing fluid or pus. The areas most commonly affected are the underarms, under the breasts, and groin. Scar tissue remains after healing. Staphylococci and Streptococci, in particular S. aureus, have been reported to be the most prevalent bacteria

The term “paronychia”, as used herein, refers to a health condition characterized by a nail infection that is an often tender bacterial or fungal infection of the hand or foot, where the nail and skin meet at the side or the base of a finger or toenail. The infection can start suddenly (acute paronychia) or gradually (chronic paronychia). It has been reported to be commonly caused by Staphylococcus aureus and Streptococcus pyogenes bacteria.

The expression “infection in atopic dermatitis”, as used herein, refers to refers to a health condition characterized by a skin lesion due to atopic dermatitis that is infected by bacteria. Generally, bacteria that cause this type of infection are from the genus Staphylococus and Streptococus, in particular from the species Staphylococcus aureus, Streptococcus pyogenes and Pseudomonas aeruginosa. Infection is in part due to the breaks in the skin resulting from the atopic dermatitis, i.e. very dry, split skin and from scratching the itchy areas. Additionally, the immunological profile of atopy favors colonization by these bacteria, which are present in most patients with atopic dermatitis, even in the absence of skin lesions. In case infection of an atopic dermatitis lesion as just defined, occurs simultaneously with, or after the treatment of, another infection (caused by the same bacteria, or by another microorganism, such as another bacteria, a virus or fungi), it is referred herein as a “superinfection in atopic dermatitis”. In a particular embodiment, said superinfection is caused by the bacteria indicated above in the definition of “infection in atopic dermatitis”.

The expression “ocular infection”, as used herein, refers to a health condition characterized by an infection that affects any part of the eye ball or surrounding area. In a particular embodiment said infection is a bacterial infection as defined above. Most common ocular infections include conjunctivitis and staphylococcal blepharitis. Thus, in a particular embodiment, the ocular infection is conjunctivitis. In another particular embodiment, the ocular infection is staphylococcal blepharitis.

The term “conjunctivitis”, or “pink eye”, as used herein, refers to a health condition characterized by inflammation of the conjunctiva of the eye. It can be caused by a bacterial infection, viral infection, allergy, eye trauma, or a foreign body in the eye. In a particular embodiment, conjunctivitis is caused by a bacterial infection. Bacterial conjunctivitis causes the rapid onset of conjunctival redness, swelling of the eyelid, and a sticky discharge, especially after sleep, which may be opaque, greyish or yellowish. Common bacteria responsible for bacterial conjunctivitis are bacteria from the genus Staphylococcus (such as S. aureus), Streptococcus (such as S. pneumoniae), Pseudomonas (such as P. auruginosa) Haemophilus species. Less commonly, Chlamydia spp. (such as Chlamydia trachomatis), Moraxella, Neisseria gonorrhoeae, β-hemolytic streptococci, or Corynebacterium diphtheria.

The expression “staphylococcal blepharitis”, as used herein, refers to a type of blepharitis caused by bacteria from the genus staphylococcus, wherein blepharitis refers to refers to a health condition characterized by an inflammation of the eyelids in which they become red, irritated and itchy and dandruff-like scales form on the eyelashes. In most cases, staphylococcal blepharitis is caused by S. aureus.

The expression “infection from the urinary tract”, as used herein, refers to a health condition characterized by an infection of the urinary tract, which includes kidneys, bladder, ureters, and urethra. When it affects the urethra it is known as urethritis. When it affects the bladder, it is known as cystitis. When it affects the kidney, it is known as pyelonephritis. It can be caused by bacteria, virus or yeast, although in most cases, it is caused by bacteria. Gram-negative bacteria such as Escherichia coli (most commonly), Proteus vulgaris, Pseudomonas aeruginosa, and Klebsiella pneumoniae cause most bladder and urethra infections. Gram-positive pathogens associated with urinary tract infection include the coagulase-negative Staphylococcus saprophyticus, Staphylococcus aureus, Enterococcus faecalis, and Streptococcus agalactiae.

The expression “infection of cardiac valves”, or “infective endocarditis”, as used herein, refers to a health condition characterized by an infection of the inner surface of the heart, in particular the valves. It is typically caused by bacteria. Bacteria that commonly cause infective endocarditis include Staphylococcus aureus, Staphylococcus epidermidis, Streptococcus viridans and coagulase negative staphylococci. The viridans group include S. oralis, S mitis, S. sanguis, S. gordonii and S. parasanguis. In some cases, it is caused by bacteria from the genus Pseudomonas, in particular by P. auruginosa. The term “infection of artificial cardiac valves”, or “Prosthetic valve endocarditis” as used herein, refers to a severe form of infective endocarditis which involves a prosthetic valve and that accounts for 20% of all cases of infective endocarditis. The most likely pathogenic mechanisms in prosthetic valve endocarditis are intraoperative contamination and postoperative infections at extracardiac sites. There is increased risk with reoperative surgery, often due to difficulties in clearing infection because of prosthetic material in place. Most common bacteria causing infection of artificial cardiac valves include those indicated above for infective endocarditis.

The expression “bone infection” or “osteomyelitis”, as used herein, refers to a condition or disease, wherein a microorganism, such as bacteria, fungi or virus, invades a bone. In children, bone infections most commonly occur in the long bones of the arms and legs. In adults, they usually appear in the hips, spine, and feet. A bone infection may result from blood stream spread of a microorganism that previously infected another region of an organism. The most common cause of bone infection is S. aureus bacteria. It can also be caused by Pseudomonas, in particular by P. auruginosa. In a particular embodiment, bone infection is caused by bacteria, in particular by one of the aforementioned bacteria.

The expression “joint infection”, “septic arthritis” or “infectious arthritis”, as used herein, refers to a health condition characterized by an infection of a tissue and/or biological liquid of a joint (such as cartilage, synovial membrane, ligaments, tendons, bursas, synovial fluid, meniscus). It can be cause by bacteria, viruses, fungi or parasites. Most commonly, joints become infected via the blood stream but may also become infected via trauma or an infection around the joint. Joint infection is most often caused by bacteria, in particular by bacteria from the genus Staphylococcus, such as S. aureus or coagulase-negative Staphylococci, Streptococcus, such as S. pyogenes, S. pneumoniae, or group B streptococci, Pseudomonas, such as P. aeruginosa, Salmonella or Brucella, or from the species E. coli, or Neisseria gonorrhoeae, Neisseria meningitides, or M. tuberculosis. In a particular embodiment, joint infection is caused by bacteria, preferably from one of the aforementioned bacteria.

The expression “infection of a wound”, as used herein, refers to a health condition characterized in that pathogenic microorganisms have grown and/or are growing in a wound. Said pathogenic microorganisms include bacteria, virus, fungi and parasites. In a particular embodiment, it refers to a wound infected by bacteria. A wound can be infected by any of the bacteria present in the environment, and thus include any of the bacteria cited in the different aspects of the invention. In preferred embodiment, the wound infection is caused by S. aureus or P. aeruginosa. When the wound consists on a cut or incision in the skin made by a practitioner, usually with a scalpel during surgery, or as the result of a drain placed during surgery, it is herein referred to as “surgical wound”. Thus, in a particular embodiment, the wound infection is a surgical wound infection. Bacterial causing said infection are any of those mentioned above for a wound infection.

The expression “infection of a burn”, as used herein, refers to a health condition characterized in that pathogenic microorganisms are have grown and/or are growing in a burn. Said pathogenic microorganisms include bacteria, virus, fungi and parasites. In a particular embodiment, it refers to a burn infected by bacteria. It can be infected by any of the bacteria present in the environment, and thus bacteria that can cause an infection of a burn include any of the bacteria cited in the different aspects of the invention. In preferred embodiment, the wound infection is caused by S. aureus or P. aeruginosa.

In a particular embodiment, bacterial infections caused by S. aureus include bacterial vaginosis, mastitis, otitis, impetigo, ecthyma, erythema, erysipelas, bacterial cellulitis, folicullitis, furunculosis, hydrosadenitis, paronychia, infection in atopic dermatitis, superinfection in atopic dermatitis, ocular infection, conjunctivitis, staphylococcal blepharitis, infection from the urinary tract, infection of cardiac valves, infection of artificial cardiac valves, bone infection, joint infection, wound infection, and a burn infection.

In a particular embodiment, bacterial infections caused by P. aeruginosa include bacterial vaginosis, mastitis, otitis, impetigo, ecthyma, erythema, erysipelas, bacterial cellulitis, folicullitis, furunculosis, hydrosadenitis, paronychia, infection in atopic dermatitis, superinfection in atopic dermatitis, ocular infection, conjunctivitis, staphylococcal blepharitis, infection from the urinary tract, infection of cardiac valves, infection of artificial cardiac valves, bone infection, joint infection, wound infection, and a burn infection. In a preferred embodiment, they include bacterial vaginosis, otitis, infection in atopic dermatitis, superinfection in atopic dermatitis, ocular infection, conjunctivitis, infection from the urinary tract, infection of cardiac valves, infection of artificial cardiac valves, bone infection, joint infection, wound infection, and a burn infection

In a particular embodiment, the biomaterial of the invention, or the biomaterial obtained by the method of the invention, is for use in the prevention of any of the aforementioned conditions or diseases. In a particular embodiment, said medical use is also herein referred when using the expression “medical use of the invention”.

As used herein “preventing” or “prevention” refers to any methodology where the health condition or disease does not occur due to the actions of the methodology (such as, for example, administration of the biomaterial of the invention). In one aspect, it is understood that prevention can also mean that the disease or condition is not established to the extent that occurs in untreated controls. For example, there can be a 5, 10, 15, 20, 25, 30, 35, 40, 50, 60, 70, 80, 90, or 100% reduction in the establishment of the disease or condition frequency relative to untreated controls. Accordingly, prevention of a disease or condition encompasses a reduction in the likelihood that a subject will develop the disease or condition, relative to an untreated subject (e.g. a subject who does not receive the biomaterial of the invention).

The medical uses of the invention comprise the administration of a therapeutically effective amount of the biomaterial of the invention, or of the biomaterial obtained by the method of the invention.

The term “therapeutically effective amount”, as used herein, refers to the sufficient amount of the biomaterial of the invention, or to a sample of said biomaterial of the size required for the biomaterial to provide the desired effect. It will generally be determined by, among other causes, the characteristics of the probiotics comprised in the biomaterial and the therapeutic effect to be achieved. It will also depend on the subject to be treated, the severity of the disease or health condition suffered by said subject, the chosen size and dose form, etc. For this reason, the doses mentioned in this invention must be considered only as guides for the person skilled in the art, who must adjust the doses depending on the aforementioned variables. In an embodiment, the effective amount produces the amelioration of one or more symptoms of the disease or condition that is being treated, for example, the reduction of the amount of pathogenic microorganism or bacteria present in the infected tissue or body region of the subject, or a reduction in the growth rate of said pathogens in said tissue or body region.

The term “subject” is used herein to describe a human or an animal. In the context of the embodiments of the present products, formulations, and processes, “subject” denotes a mammal, such as a human, to whom the biomaterial are administered. As it will be understood, when the disease to be treated is related to female organs, the subject to be treated is a female. Thus, when the bacterial infection referred in the medical use of the invention is bacterial vaginosis, the subject is a female.

The biomaterial of the invention or the biomaterial obtained by the method of the invention has to be formulated so that, once administered, the probiotics of the biomaterial of the invention, or the modifications said probiotics do in their external media, affect the tissue or body region to be treated.

In some embodiments, the biomaterial of the invention is administered topically.

The term “topical administration”, as used herein, refers to the application of a product on a body surfaces such as the skin or mucous membranes. The term “mucous membrane” or “mucosa”, has been defined above. Thus, topical administration, as used herein, refers to the local administration in a region of the skin or of any of the tissues referred in the definition of “mucosa” above.

In some embodiment, the biomaterial of the invention is administered locally in the infected body region or tissue. In a particular embodiment, said body region nor tissue is a mucosa, a soft tissue, a bone, a joint or a tissue in contact with a prosthesis.

The term “joint”, as used herein, refers to the area where two bones are attached for the purpose of permitting body parts to move. It is also known as articulation. It is commonly formed by cartilage, ligaments, tendons, synovial membrane, bursas, synovial fluid, and/or meniscus. Thus, as understood by a skilled person, when the biomaterial of the invention or obtained by the method of the invention is administered locally in the joint, it is applied to any of the elements of the joint just indicated.

The biomaterial is thus preferably in the form of a patch, to be applied in the infected body region or tissue area. The term “patch”, as used herein, refers to an adhesive medicated product that is placed on the skin or mucosa of a subject, so to deliver a specific dose of therapeutically active component through the skin and into the bloodstream. Generally, the patch provides a controlled release of the therapeutically active component into the patient.

The expression “therapeutically active component”, as used herein, refers to the component of a medicament, product, or pharmaceutical composition that directly or indirectly elicits the therapeutic activity of said medicament, product or composition when administered to a subject in need thereof. It thus does not include carriers, diluents, vehicles, adjuvants or the like.

In a particular embodiment, the therapeutically active component of the biomaterial of the invention or of the biomaterial obtained by the method of the invention are the probiotics comprised in said biomaterial. In another particular embodiments it is the components liberated by said probiotics. As well-known by an expert in the field, several probiotics, including lactic acid bacteria, such as bacteria from the genus Lactobacillus (in particular the species of Lactobacillus specified in any of the aspects above), are known for their capacity to liberate lactic acid in the medium. Lactic acid decreases the pH of the media. For instance, as shown in Example 3 below, bacteria from the genus Lactobacillus can drop the pH of their surrounding media from pH 7 to pH 4. Thus, in a particular embodiment, the therapeutically active component of the biomaterial of the invention or of the biomaterial obtained by the method of the invention the lactic acid excreted by the probiotics comprised in said biomaterial.

The size of the patch can vary with the size of the infection, the dose required, or the area to be treated. In a particular embodiment, the patch has an area of 1 m², 750 cm², 500 cm², 400 cm², 300 cm², 200 cm², 100 cm², 90 cm², 80 cm², 70 cm², 60 cm², 50 cm², 40 cm², 30 cm² 25 cm², 20 cm², 15 cm², 12 cm², 10 cm², 8 cm², 5 cm², 4 cm², 2 cm², 1 cm², 0.5 cm², preferably, of 12 cm² In another particular embodiment, the thickness is of 0.1 mm, 0.2 mm, 0.5 mm, 0.7 mm, 0.8 mm, 0.9 mm, 1 mm, 1.2 mm, 1.5 mm, 1.7 mm, 2 mm, 2.2 mm, 2.5 mm, 2.7 mm, 3 mm, 3.5 mm, 4 mm, 4.5 mm, 5 mm, 6 mm, 7 mm, 8 mm, 9 mm, 1 cm, 1.5 cm, 2 cm, 2.5 cm, 3 cm, 3.5 cm, 4 cm, 5 cm, 6 cm, 7 cm, 8 cm, 9 cm 10 cm, preferably of 1.5 mm. In another particular embodiment, the patch is circular, rectangular, square, star-shaped, or with an irregular shape. In a preferred embodiment, it has a circular shape.

Said patch may be provided with no additional substances or with a pharmaceutically acceptable carrier. Thus, in a particular embodiment, the invention relates to a pharmaceutical composition, comprising the biomaterial of the invention, and a pharmaceutically acceptable carrier. As understood by a skilled person, in a particular embodiment the biomaterial of the pharmaceutical composition of the invention may be formulated as a patch. In another particular embodiment, the invention relates to the pharmaceutical composition of the invention for use in medicine. In another particular embodiment, the invention relates to the pharmaceutical composition of the invention for use in the treatments specified in the present aspect of the invention. In a particular embodiment, said medical uses are also herein referred when using the expression “medical uses of the invention”.

The term “pharmaceutical product” is understood in its widely meaning in this description, including any composition that comprises an active ingredient, in this case, the strains of the invention preferably in form of composition, together with pharmaceutically acceptable excipients. The term “pharmaceutical product” is not limited to medicaments. The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. Suitable carriers, excipients, etc. can be found in standard pharmaceutical texts.

The expression “pharmaceutical composition”, as used herein, is understood in its widely meaning in this description, including any composition that comprises an active ingredient, in this case, the probiotics of comprised in the biomaterial invention or even the biomaterial of the invention, preferably in form of composition, together with pharmaceutically acceptable excipients. The term “pharmaceutical composition” is not limited to medicaments. The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. The expression “Pharmaceutically acceptable carrier”, or “pharmaceutically acceptable excipient”, as used herein, refers to a therapeutically inactive substance to be used for incorporating the active ingredient and which is acceptable for the patient from a pharmacological toxicological point of view and for the pharmaceutical chemist who manufactures it from a physical/chemical point of view with respect to the composition, formulation, stability, acceptation of the patient and bioavailability.

The number and the nature of the pharmaceutically acceptable excipients depend on the desired dosage form. The pharmaceutically acceptable excipients are known by the person skilled in the art (Fauli y Trillo C. (1993) “Tratado de Farmacia Galenica”, Luzan 5, S.A. Ediciones, Madrid). Said compositions can be prepared by means of the conventional methods known in the state of the art (“Remington: The Science and Practice of Pharmacy”, 20th edition (2003) Genaro A. R., ed., Lippincott Williams & Wilkins, Philadelphia, US).

In a particular embodiment, the therapeutically active component of the pharmaceutical composition comprise, essentially comprise, or consists of the probiotics comprised in the biomaterial of the invention, or in the biomaterial obtained by the method of the invention, and the pharmaceutically acceptable excipient comprise, essentially comprise, or consists of the BC of the invention.

The biomaterial referred in the medical uses of the invention, as well as the pharmaceutical composition of the invention, may also be formulated as a crème, or ointment. As it will be understood by a skilled person, in this case the biomaterial is provided in the form of several microparticles, comprising each microparticle the BC and the probiotics entrapped in it. When provided in said form, as well as when provided in the form of a patch, it can comprise an oily substance originating from vegetable, marine or animal sources. Suitable liquid oil includes saturated, unsaturated or polyunsaturated oils. By way of example, the unsaturated oil may be olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils or mixtures thereof, in any proportion. These creams or ointments may further comprise poly-unsaturated fatty acids. In one or more embodiments, said unsaturated fatty acids are selected from the group of omega-3 and omega-6 fatty acids. Examples of such polyunsaturated fatty acids are linoleic and linolenic acid, gamma-linoleic acid (GLA), eicosapentaenoic acid (EPA) and docosahexaenoic acid (DHA). Such unsaturated fatty acids are known for their skin-conditioning effect, which contribute to the therapeutic benefit of the composition. Thus, the composition can include at least 6 percent of an oil selected from omega-3 oil, omega-6 oil, and mixtures thereof. Also usable are the essential oils, which are also considered therapeutically active oils, which contain active biologically occurring molecules and, upon topical application, exert a therapeutic effect, which is conceivably synergistic to the beneficial effect of the probiotic mixture in the composition. Another class of therapeutically active oils includes liquid hydrophobic plant-derived oils, which are known to possess therapeutic benefits when applied topically. Silicone oils also may be used and are desirable due to their known skin protective and occlusive properties. Suitable silicone oils include non-volatile silicones, such as polyalkyl siloxanes, polyaryl siloxanes, polyalkylaryl siloxanes and polyether siloxane copolymers, polydimethylsiloxanes (dimethicones) and poly(dimethylsiloxane)-(diphenyl-siloxane) copolymers. These are chosen from cyclic or linear polydimethylsiloxanes containing from about 3 to about 9, preferably from about 4 to about 5, silicon atoms. Volatile silicones such as cyclomethicones can also be used. Silicone oils are also considered therapeutically active oils, due to their barrier retaining and protective properties.

The composition may also be in the form of a capsule or tablet, in particular when addressed to vaginal, oral or anal administration. In this case, the biomaterial referred in the medical uses of the invention or in the pharmaceutical composition of the invention, may be in the form of several microparticles as well. The term “capsule” refers to a hard shell pharmaceutical capsule. The capsule consists of a body and cap and may comprise a fill formulation containing the probiotic composition. Capsules suitable for use according to the invention include, without limitation NPcapsCR′ available from Capsugel which contain pullulan, carageenan and potassium chloride, as well as capsules described in U.S. Pat. No. 8,105,625 and US Patent Application Publication No. 2005/0249676. In one aspect, capsules for use according to the invention comprise pullulan with a molecular weight between about 50 to 500 kDa, between 100 to 400 kDa, between about 150 to 300 kDa and preferably between about 180 and 250 kDa. In another aspect, capsules for use according to the invention comprise pullulan from about 50 percent to about 100 percent by weight (unfilled capsule). In other aspects, the capsules comprise about 60 to 90 or 70 to 90, or 80 to 90 wt percent pullulan. Preferably the capsules comprise about 85 to 90 wt percent pullulan. Capsules for use according to the invention may further comprise (in addition to pullulan) one or more gelling agents (e.g. hydrocolloids or polysaccharides such as alginates, agar gum, guar gum, carob, carrageenan, tara gum, gum arabic, pectin, xanthan and the like); salts comprising cations such as K, Li, Na, NH4, Ca, Mg; and/or surfactants such as sodium lauryl sulphate, dioctyl sodium sulfosuccinate, benzalkonium chloride, benzethonium chloride, cetrimide, fatty acid sugar esters, glycerl monooleate, polyoxyethylene sorbitan fatty acid esters, polyvinylalcohol, dimethylpolysiloxan, sorbitan esters or lecithin.

Capsules for use according to the invention may further comprise one or more plasticizing agents (e.g. glycerol, propylene glycol, polyvinyl alcohol, sorbitol, maltitol and the like); dissolution enhancing agents (e.g. maltose, lactose, sorbitol, mannitol, xylitol, maltitol and the like); strengthening agents (e.g. polydextrose, cellulose, maltodextrin, gelatin, gums and the like); colorants, and/or opacifiers as described in U.S. Pat. No. 8,105,625. In a preferred embodiment, the capsule comprises pullulan in an amount of 85 percent to 90 percent by weight, potassium chloride in an amount of 1.0 percent to 1.5 percent by weight, carrageenan in an amount of 0.1 percent to 0.4 percent by weight, one or more surfactants in an amount of 0.1 percent to 0.2 percent by weight and water in an amount of 10 percent to 15 percent by weight.

In some embodiments, the biomaterial referred in the medical uses of the invention and in the pharmaceutical composition of the invention is supplied as a powder, i.e. in the form of several microparticles, and can be administered by a suitable applicator. In this case, the biomaterial formulation may be supplied as a component of a kit, which includes an applicator. The formulation may be pre-packaged in the applicator, or supplied as a separate item of the kit. In some other embodiments, the biomaterial is incorporated into vaginal tampons, or suppository. A kit comprising the tampons or suppositories optionally includes one or more applicators. Suitable dosage forms also include vaginal suppositories, including capsules and tablets, which can be administered by with or without a suitable applicator. The choice of the dosage form depends on a variety of factors. For example, the chosen dosage form should ensure stability of the formulation's ingredients during storage, convenient administration and quick delivery of the formulation in the environment of the cavity where it is to be applied. In particular, the dosage form of the formulations should not detrimentally affect viability or allow premature (prior to administration) reconstitution of the probiotic components of the formulation. To this end, the dosage form should not contain water. At the same time, the dosage form should allow for quick dispersion or dissolution of the formulation's ingredients in the environment of the cavity upon administration. For example, if a tablet or a capsule is chosen as a dosage form, it should be formulated to disintegrate quickly when administered into the desired body cavity.

Additionally, the administration can be chronic or intermittent, as deemed appropriate by the supervising practitioner, particularly in view of any change in the disease state or any undesirable side effects. “Chronic” administration refers to administration of the composition in a continuous manner while “intermittent” administration refers to treatment that is done with interruption.

For instance, the biomaterial referred in the medical uses of the invention or the pharmaceutical composition of the invention may be administered for at least 1 day, at least 3 days, at least 6 days, or as prescribed by a physician or until the improvement or reduction of the symptoms is achieved, in particular, a reduction of the infection as defined above.

In a particular embodiment, the biomaterial referred in the medical uses of the invention or the pharmaceutical composition of the invention may be administered in an amount of 1-5 doses per day, preferably 1 dose per day. It can also include 1 dose every 2 days, every 3 days, every 4 days, every 5 days, every 6 days, every week, every 10 days, or every 2 weeks. In another particular embodiment, it is administered in said amount during any of the periods indicated just above.

The effective amount of colony forming units (CFU) for the probiotic strains in the composition to be administered will be determined by the skilled in the art and will depend upon the final formulation. For instance, when administered orally without any other active agent, the total amount of the probiotics present in a single dose of the biomaterial or composition is that giving an effective daily dose of from 10⁷ to 10¹² CFU, according to the current legislation, preferably from 10⁹ to 10¹¹ CFU. When administered as a patch, vaginally or rectally, said amount is that giving an effective daily dose of from 10³ to 10¹² CFU, preferably from 105 to 100 CFU. The term “colony forming unit” (“CFU”) is defined as number of bacterial cells as revealed by microbiological counts on agar plates. Food supplements usually contain probiotic strains in an amount ranging from 10⁷ and 10¹² CFU/g. In a particular embodiment, the composition of the invention is a food supplement for daily doses comprising between 10⁹-10¹¹ CFU/g.

In a particular embodiment, the definitions and embodiments provided in any of the aspects above apply to the medical uses of the invention and to the pharmaceutical composition of the invention.

5. Coated Food Product and Use of the Biomaterial of the Invention as a Coat in a Coated Food Product.

The authors of the present invention have observed that the biomaterial of the invention containing BC and probiotics shows antibacterial activity against bacteria that are commonly found in hospitals (in particular against S. aureus and P. aeruginosa) and that once introduced in the organism of a subject, or accidentally ingested by a subject, can cause infectious diseases (see example 4). Thus, by virtue of being a solid biomaterial, the biomaterial of the invention can be advantageously used for the preparation of coats that allow maintaining medical devices and edible compositions under sterile conditions, until they are put in contact with an organism in the case of medical devices, or ingested in case of edible compositions.

Thus, a seventh aspect of the invention relates to a coated food product which comprises:
(i) a biomaterial according to the first or third aspect of the invention, and
(ii) an edible filling composition,

wherein the biomaterial (i) coats the filling composition (ii).

Said coated food product is herein referred to as the coated food product of the invention.

An additional aspect, the invention relates to the use of the biomaterial of the first or third aspect of the invention as a coat in a coated food product.

Said use is herein referred to as the first use of the invention.

The term “food product”, as used herein refers to any product that is edible by a subject. The term subject has been defined above, and refers to an animal, preferably a human. The term “edible”, as used herein refers to a product that can be chewed, and swallowed, or directly swallowed, and that is non-toxic for the aforementioned subject. Food products comprise any of the products described in the embodiments bellow in connection with the edible filing composition of the coated food product of the invention.

The term “coated food product”, as used herein, refers to any food product that comprises a coat. Said food product is thus composed of a coat and an edible filling composition.

The term “coat”, as used herein refers to a packaging with barrier properties, that reduce gases and water vapor exchanges between the food and the surrounding environment, decreasing the rates of chemical, physical and microbiological changes. In some cases, the coat may also comprise chemical or biological properties that contribute to decreases the aforementioned changes. Therefore, generally the coat extends food stability and assures its quality/safety during shelf life. Transparency of the coat is also a characteristic that can be desirable for a coated food product, so that the consumer can see the food product and its aspect.

The term “filling”, “filling composition”, or “edible filling composition”, as used herein, refers to the edible product that is surrounded by the coat in the food product. In some embodiments, the filling composition comprises animal matter, vegetables, cereals, fruits or combinations thereof. In a particular embodiment, it also comprises juices and/or syrups. In another particular embodiment, it also comprises an additive or several additives accepted by the corresponding food security agency, such as the European Food Safety Authority (EFSA), the US Food and drug Agency (FDA), or by the World Health Organization (WHO).

The term “animal matter”, as used herein, refers to any product, preferably meat, derived from an animal. Said animal can be American bison, carabao, cattle, water buffalo, domesticated yak, springbok, greater kudu, gemsbok, impala, alpaca, llama, camel, Dog (Kuro, Poi dog, Nureongi, Xoloitzcuintle), coat, moose, reindeer, red deer, fallow deer, elk, cat, donkey, horse, rabbit, hare, kangaroo, sheep, guinea pig, edible dormouse, coypu, capybara, rat, domestic pig, wild boar, frog, chicken, Cornish game hen, duck, goose, turkey, quail, pigeon, guineafowl, ostrich, or emu.

In a particular embodiment, the filling composition comprises animal matter and said animal matter comprises red meat, meat from pork, poultry, fish or combinations thereof.

The term “red meat”, as used herein, refers to the term commonly known by an expert in the field. Non-limiting examples of red meat include meat from beef, lamb, goat, bison, horse, venison, etc.

As used herein, the term “poultry”, refers to the term commonly known by an expert in the field. Non-limiting examples of poultry animals include chicken, Cornish game hen, duck, goose, turkey, quail, pigeon, guineafowl, ostrich, or emu.

In a particular embodiment, meat from fish includes meat from any fish usually used in the diet of a subject, preferably humans. Non-limiting examples of said fish include anchovies, barracuda, Basa, Bass, Black cod/Sablefish, Blowfish, Bluefish, Bombay duck, Bream, Brill, Butter fish, Catfish, Cod, Dogfish, Dorade, Eel, Flounder, Grouper, Haddock, Hake, Halibut, Herring, Ilish, John Dory, Lamprey, Lingcod, Mackerel, Mahi, Monkfish, Mullet, Orange roughy, Parrotfish, Patagonian toothfish, Perch, Pike, Pilchard, Pollock, Pomfret, Pompano, Sablefish, Salmon, Sanddab, particularly Pacific sanddab, Sardine, Sea bass, Shad, Shark, Skate, Smelt, Snakehead, Snapper, Sole, Sprat, Sturgeon, Surimi, Swordfish, Tilapia, Tilefish, Trout, Tuna, Turbot, Wahoo, Whitefish, Whiting, Witch, Whitebait. It also includes shellfish such as crab, crayfish, langostino, lobster, shrimp, cockle, cuttlefish, clam, loco, mussel, octopus, oyster, periwinkle, scallop, squid, conch, nautilus. It also includes fish roe, such as Caviar, Ikura, Kazunoko, lumpfish roe, masago, shad roe, tobiko.

In another particular embodiment, the animal matter also refers to products from insects, including chapulines, aguey worm, mopane worm, silkworm, locust, grasshopper.

In a particular embodiment, the animal matter refers to processed meat. The expression “processed meat”, as used herein refers to any meat which has been modified in order either to improve its taste or to extend its shelf life. Methods of meat processing include salting, curing, fermentation, and smoking. Processed meat is usually composed of pork or beef, but also poultry, while it can also contain offal or meat by-products such as blood. Processed meat products include bacon, ham, sausages, salami, corned beef, jerky, canned meat and meat-based sauces. As used herein, processed meat also refers to modified meat as defined herein, from fish, such as for instance, surimi. Meat processing includes all the processes that change fresh meat with the exception of simple mechanical processes such as cutting, grinding or mixing. In a particular embodiment, the processed meat comprises any of the meat from animals and/or fish indicated above. In a preferred embodiment, it refers to imitations of the meat obtained from any of said animals.

The coated food product is preferably a coated moulded food product, in which the ingredients have been processed (e.g. by chopping, shredding or grinding the ingredients). Coated moulded food products include burgers, kebabs and sausages. In preferred embodiments, the coated food product is a sausage, such as a meat sausage. Skinless meat sausages are particularly preferred.

The term “vegetables”, as used herein refers to the term commonly known by the expert in the field. In particular, it refers to the parts of plants that are edible by a subject, such as an animal, preferably a mammal, more preferably a human. Non-limiting examples of said plant products include leaves, stems, flower, roots, seeds sprouts, pods, tubers, bulbs and combinations thereof. Non-limiting examples of said plants include plants from the species Brassica oleracea, Brassica rapa, Raphanus sativus, Daucus carota, Pastinaca sativa, Beta vulgaris, Lactuca sativa, Phaseolus vulgaris, Phaseolus coccineus, Phaseolus lunatus, Vicia faba, Pisum sativum, Solanum melongena, salanum lycopersicum, Cucumis sativus, Cucurbita spp., Allium cepa, Allium sativum, Allium ampeloprasum, Capsicum annuum, Spinacia oleracea, Dioscorea spp., Ipomoea batatas Manihot esculenta, or asparagus officinalis. In a particular embodiment, the vegetables are cooked. In another particular embodiment, the vegetable are raw. In another particular embodiment, the term refers to mashed, fresh and/or lyophilized vegetables.

The term “cereal” as used herein, refers to the term commonly by an expert in the field. In particular, it refers to the edible content of the grain, or caryopsis, of specific grass. Non-limiting examples of grass form which the cereals are obtained include grass form the species Zea mays, Oryza glaberrima, Oryza sativa, Hordeum vulgare, Eleusine coracana, Eragrostis tef, Panicum miliaceum, Panicum sumatrense, Pennisetum glaucum, Setaria italica, Digitaria exilis, Digitaria iburua, Digitaria compacta, Digitaria sanguinalis, Echinochloa esculenta, Echinochloa frumentacea, Echinochloa oryzoides, Echinochloa stagnina, Echinochloa crus-galli, Paspalum scrobiculatum, Brachiaria deflexa, Urochloa ramosa, Coix lacryma-jobi, Avena sativa, Secale cereale. It also includes grass from the genus Triticum, in particular from the species T. aestivum, Sorghum, in particular from the S. bicolor, or Digitaria, in particular from the species D. exilis or D. iburua. It also includes the hybrid Triticale, from Triticum and Secale. In a particular embodiment, the cereals are cooked. In another particular embodiment, the cereals are raw. In another particular embodiment, the term refers to mashed, fresh and/or lyophilized cereals.

The term “fruit” as used herein, refers to term commonly known by an expert in the field. In particular, it refers to the seed-associated structures of a plant that are sweet or sour, and edible in the raw state. Non-limiting examples of fruit include apples, pears, bananas, grapes, lemons, oranges or strawberries.

The coated food product may be a raw, partially cooked or cooked food product.

The animal matter, vegetables, cereals, fruits or combinations thereof of the filling composition, may be used in the filling composition in an amount of at least 20% by weight, preferably at least 25% by weight, and more preferably at least 30% by weight of the filling composition. Said edible products may be used in a total amount of up to 60% by weight, preferably up to 50% by weight, and more preferably up to 45% by weight of the filling composition. Thus, the filling composition may comprise animal matter, vegetables, cereals, fruits or combinations thereof in a total amount of from 20 to 60% by weight, preferably from 25 to 50% by weight, and more preferably from 30 to 45% by weight of the filling composition. In a preferred embodiment, the filling composition comprises animal matter in any of the aforementioned % by weight. In a particular embodiment, the filling composition comprises vegetables in any of the aforementioned % by weight. In another particular embodiment, the filling composition cereals in any of the aforementioned % by weight. In another embodiment, the filling composition comprises fruits in any of the aforementioned % by weight.

Water may be used in filling composition in an amount of at least 30% by weight, preferably at least 35% by weight, and more preferably at least 40% by weight, by weight of the filling composition. Water may be used in an amount of up to 60% by weight, preferably up to 55% by weight, and more preferably up to 50% by weight of the filling composition. Thus, the filling composition may comprise water in an amount of from 30 to 60% by weight, preferably from 35 to 55% by weight, and more preferably from 40 to 50% by weight of the filling composition. It will be appreciated that these amounts relate to the water that is added to the filling composition during its preparation, and do not include water that has been added to the filling composition as part of the animal matter, vegetables, cereals, fruits or combinations thereof.

In a preferred embodiment, the edible filling composition is as the filling composition defined in patent application GB2570934A. In a particular embodiment, the animal matter of said composition is as defined herein. In another particular embodiment, the filling composition is as defined in GB2570934 wherein the expression “animal matter”, is substituted by vegetables, cereals, fruits or combinations thereof, where said terms are as defined herein.

Thus in a particular embodiment, the filling composition comprises animal matter, water, protein extender, starch (modified and, if used, unmodified) and fibre in a combined total amount of at least 80% by weight, preferably at least 90% by weight, and more preferably at least 95% by weight of the filling composition. In another particular embodiment, the filling composition comprise animal matter, vegetables, cereals, fruits or combinations thereof water, protein extender, starch (modified and, if used, unmodified) and fibre in a combined total amount of at least 80% by weight, preferably at least 90% by weight, and more preferably at least 95% by weight of the filling composition.

The modified and unmodified starch, the protein extender and the fiber of the filling composition are as defined in GB2570934A.

In a particular embodiment, the definitions and embodiments provided in any of the aspects above apply to the coated food product of the invention and to the first use of the invention.

6. Packaged Medical Device and Use of the Coated and Use of the Biomaterial of the Invention for the Packaging of a Medical Device.

An eighth aspect relates to a packaged medical device wherein the device is packaged in a container which comprises a biomaterial of the first or third aspect of the invention.

Said medical device is herein referred to as the medical device of the invention.

Another aspect of the invention relates to the use of a biomaterial of the first or third aspect of the invention for the packaging of a medical device.

Said use is herein referred to as the second use of the invention.

The expression “medical device”, as used herein, refers to any instrument, apparatus, appliance, material, or other article—whether used alone or in combination—to be used in a medical intervention, such as a surgical intervention, an exploration intervention, or a diagnosis test.

In a particular embodiment, medical device refers to instruments that cause or can cause a trauma in a tissue of the subject being intervened, or that are placed within an organ or tissue of a subject, after surgery. Thus, in a preferred embodiment, the medical device of the invention and referred in the second use of the invention is a surgical device. In another embodiment, the medical device of the invention and referred in the second use of the invention are prosthesis. In another particular embodiment, the medical device of the invention and referred in the second use of the invention is a catheter.

The term “surgical instrument”, or “surgical device”, as used herein, refers to a tool or device for performing specific actions or carrying out desired effects during a surgery or examination, such as modifying biological tissue, or to provide access for viewing it. Non-limiting examples of surgical instruments include grasper (such as forceps), clamps and occluders, clamps and occluders for blood vessels, needle drivers or needle holders (used to hold suture needle while it is passed through tissue and to grasp suture while instrument knot tying), retractors (used to spread open skin, ribs and other tissue), distractors, positioners, stereotactic devices, scalpels, lancets, drill bits, rasps, trocars, ligasure, harmonic scalpel, surgical scissors, rongeurs, dilators and specula (for access to narrow passages or incisions), suction tips and tubes (for removal of bodily fluids), sealing devices (such as surgical staplers), irrigation and injection needles, tips and tubes (for introducing fluid), powered devices (such as drills, cranial drills and dermatomes), scopes and probes (including fiber optic endoscopes and tactile probes), carriers and appliers for optical, electronic, and mechanical devices, catheters, ultrasound tissue disruptors, cryotomes and cutting laser guides, measurement devices (such as rulers and calipers).

The term “prosthesis”, or “prosthetic implant”, as used herein, refers to an artificial device that replaces a missing body part, organ part or organ, which may be lost through trauma, disease, or a condition present at birth (congenital disorder). Prostheses are intended to restore the normal functions of the missing body part, organ or organ part. Insertion of the prosthesis may require surgery. Non-limiting examples of prosthesis include artificial heart valves, joint prosthesis, craniofacial prosthesis or limb prosthesis.

The term “catheter”, as used herein, refers to a thin tube made from medical grade materials that can be inserted in the body to treat diseases or perform a surgical procedure. By modifying the material or adjusting the way catheters are manufactured, it is possible to tailor catheters for cardiovascular, urological, gastrointestinal, neurovascular, and ophthalmic applications. Catheters can be inserted into a body cavity, duct, or vessel. Functionally, they allow drainage, administration of fluids or gases, access by surgical instruments, and also perform a wide variety of other tasks depending on the type of catheter. Catheters include urinary catheter, pigtail catheter, artery or vein catheter, peripheral venues catheter, central venous catheter, Swan-Ganz catheter, umbilical line, Quinton catheter, intrauterine catheter, Whiz Catheter, lumbar drainage catheter.

In order to reduce the risks of infection during or after a medical intervention, medical devices, and in particular surgical devices, have to be sterilized and maintained in a sterile atmosphere until use by physicians. For that purpose, they are packaged to be maintained in an isolated and sterile atmosphere while they are not being used by physicians, i.e. during transport to the hospital, or after sterilization after having been used in a previous medical intervention.

The expression “packaged in a container”, or “packaging” as used herein, refers to the fact that the medical device is comprised in a container that serves as a barrier for microorganisms, including bacteria, so that said microorganisms cannot enter in contact with the medical device. In a particular embodiment, said container is sealed. As understood by a killer person, in this container, the atmosphere in contact with the medical device is chemically and biologically stable and microorganisms in contact with the container cannot enter inside the container. Thus, microorganism in contact with the container cannot enter in contact with the medical device.

In a particular embodiment, about 100%, 90%, 80%, 07%, 60%, 05%, 40%, 30%, 20%, 15%, 10%, 5%, 2%, 1%, preferably about 100% of the material of the container where the medical device is packaged is the biomaterial of the invention. In another embodiment, any of the above % of the material of said container is of the biomaterial obtained by the method of the invention.

In another embodiment, the biomaterial of the invention, covers about 100%, 90%, 80%, 07%, 60%, 05%, 40%, 30%, 20%, 15%, 10%, 5%, 2%, 1%, preferably about 100% of the internal surface of the container in which the medical device is packaged. In another embodiment, the biomaterial obtained by the method of the invention, covers about 100%, 90%, 80%, 07%, 60%, 05%, 40%, 30%, 20%, 15%, 10%, 5%, 2%, 1%, preferably about 100% of the internal surface of the container in which the medical device is packaged.

In another embodiment, the biomaterial of the invention covers about 100%, 90%, 80%, 07%, 60%, 05%, 40%, 30%, 20%, 15%, 10%, 5%, 2%, 1%, preferably about 100% of the surface of the container directly in contact with the atmosphere that is in contact with the medical device. In another embodiment, the biomaterial obtained by the method of the invention covers about 100%, 90%, 80%, 07%, 60%, 05%, 40%, 30%, 20%, 15%, 10%, 5%, 2%, 1%, preferably about 100% of the surface of the container directly in contact with the atmosphere that is in contact with the medical device.

In another particular embodiment, the biomaterial of the invention covers about 100%, 90%, 80%, 07%, 60%, 05%, 40%, 30%, 20%, 15%, 10%, 5%, 2%, 1%, preferably about 100% of the surface of the medical device. In another particular embodiment, the biomaterial obtained by the method of the invention covers about 100%, 90%, 80%, 07%, 60%, 05%, 40%, 30%, 20%, 15%, 10%, 5%, 2%, 1%, preferably about 100% of the surface of the medical device.

In another embodiment, the biomaterial of the invention or obtained by the method of the invention comprised within the container wherein the medical device is packaged is of any of the shapes and sizes indicated for the biomaterial of the invention.

In a particular embodiment, the container in which the medical device is packaged consists of the biomaterial of the invention. Said container is as defined above and is characterized in that the material in which it is made is the biomaterial of the invention. Any of the embodiments above related to the biomaterial of the invention comprised in the container wherein the medical device is packaged apply to the just mentioned embodiment.

As understood by a skilled person, the second use of the invention is characterized in that the biomaterial is part of, or consists of, the container where the medical device is packaged, as described in any of the embodiments above.

In a particular embodiment, the definitions and embodiments provided in any of the aspects above apply to the medical device of the invention and to the second use of the invention.

In a particular embodiment, the term consists of, essentially consists of, and comprises are interchangeable in any embodiment of any aspect of the present invention.

The invention is described below by means of the following merely illustrative and non-limiting examples of the scope of the invention.

### EXAMPLES

Materials and Methods

Bacterial Culture.

The lyophilized Acetobacter xylinum (ATCC 11142, Ax) was supplied by the Colección Española de Cultivos Tipo (CECT) and grown in Hestrin-Schramm agar (HS) (Schramm M. and Hestrin S. 1954, J. Gen. Microbiol., 11 123-129) at 30° C. Lactobacillus fermentum (Lf) and Lactobacillus gasseri (Lg) were kindly provided by Biosearch Life S.A and grown in de Man, Rogosa and Sharpe (MRS) medium (Oxoid) at 37° C.

Synthesis of Probiotic Cellulose.

The synthesis of probiotic celluloses (Lf- and Lg-cellulose) were carried out by co-culturing 0.1 mL of an Ax suspension (OD_{600 nm}=0.3) and 0.1 mL of a probiotic (Lf or Lg) suspension (OD_{600 nm}=0.4) in 1 mL of HS medium and aerobic conditions at 30° C. The material obtained after 3 days of culture is referred to as bacterial cellulose (BC). BC can be obtained by performing the culture under static conditions, or under dynamic conditions at 180-200 rpm. Afterwards, HS medium was replaced by 5 mL of MRS and BC was incubated in an anaerobic atmosphere at 37° C. for 48 hours (FIG. 4). The MRS medium was replaced after 24 hours. After 48 hours of culturing in MRS, probiotic-celluloses (Lf- and Lg-cellulose) were obtained. The same conditions were employed in the coculture of A. xylinum and B. breve. Because of B. breve is Cysteine-dependent, HS and MRS culture media were enriched with 5 μg/mL of Cys. Finally, probiotic-celluloses were collected, washed with sterile saline solution and characterized.

Gram Staining.

This staining protocol allows differentiating between two major bacterial groups, Gram-positive (stained purple) and Gram-negative (stained red) cells. Ax is a Gram-negative bacterium, whereas Lf and Lg are Gram-positive bacteria. After 1, 2 and 7 days of incubation in MRS, Lf-cellulose was dehydrated in gradient ethanol and washed with xylene (S. C. Becerra, et al., 2016, BMC Res. Notes, 9:1-10). Then, the samples were embedded in paraffin and transversally cut in 4 m sections using a microtome. Slides were deparaffinized, cleared in xylene, and rehydrated before the staining. Then, a standard Gram staining protocol was performed. In brief, crystal violet was applied for 1 minute at room temperature, and slides were briefly rinsed under running water to remove the excess of staining. Iodine mordant was applied for 30 seconds and washed with water. To remove violet crystal from Gram-negative bacteria, slides were covered with EtOH for 15 seconds and quickly rinsed under running water until the water run clear. Finally, Gram-negative bacteria were stained with safranin for 1 minute and rinsed with water. The slides were observed using an iScope (Euromex) microscope, in bright field mode and under a 100× immersion oil objective to differentiate between Gram-positive and Gram-negative. The same slides were also observed using a Nikon Eclipse E200 microscope, in dark field mode and under a 10× objective to obtain macroscopic images of the whole Lf-cellulose section. Images were acquired with an AxioCam ERc 5s (ZEISS) camera.

Field Emission Scanning Electron Microscopy (FESEM).

Probiotic celluloses were fixed in 1 mL of cacodylate buffer (0.1 M, pH 7.4) containing 2.5% of glutaraldehyde at 4° C. for 24 h. Subsequently, samples were washed with cacodylate buffer three times for 30 min at 4° C. The samples were stained with osmium tetroxide (OsO₄) solution (1% v/v) for 2 hours in the dark, being then repeatedly rinsed with Milli-Q water to remove the excess of OsO₄ solution. Samples were then dehydrated at room temperature with ethanol/water mixtures of 50%, 70%, 90% and 100% (v/v) for 20 min each, being the last concentration repeated three times and dried at the CO₂ critical point. Finally, dehydrated samples were mounted on aluminum stubs using a carbon tape, sputtered with a thin carbon film, and analyzed using a FESEM (Zeiss SUPRA40V) of the Centre for Scientific Instrumentation (University of Granada, CIC-UGR). The size (width) distribution of each condition was obtained by measuring 100 fibres of different SEM micrographs with ImageJ software (version 1.48v; NIH, Bethesda, Md.).

Quantification of Immobilized Probiotics.

Probiotic cellulose (2 cm-diameter, 1.5 mm-thick) was digested with cellulase from Trichoderma reesei (No C2730-50ML, Sigma-Aldrich). For this purpose, each sample was immersed in 2 mL of enzyme solution (50 μL cellulase/mL potassium phosphate buffer, 50 mM, pH 6) and incubated at 37° C. for 1 h, with orbital shaking (180 rpm) (Y. Hu, et al., 2011, Mater. Res. Part B Appl. Biomater., 97:114-123). Then, the samples were centrifuged to collect the probiotics and washed three times with saline solution. Probiotics were suspended in 5 mL of saline solution and colony forming units (CFU) were determined by plating in MRS-agar plates. The serial dilution with number of visible colonies around 20-300 was used to calculate CFU.

The mass of BC was weighted to denote the concentration of probiotics as CFU per milligram of cellulose. To this aim, samples were immersed in ethanol after the co-culture in HS medium (aerobiosis), boiled in deionized water for 30 min, treated with 0.1 M NaOH at 90° C. for 1 h, and washed with deionized water until neutral pH was achieved.³ With this treatment BC was purified and Ax bacteria were removed. Finally, purified celluloses were dried at 100° C. and weighted. Three replicates were measured. Following this protocol, 1.4·10¹¹ and 8.7·10¹⁰ CFU of Lf and Lg, respectively, per mg of cellulose were determined.

In Vitro Live-Dead Viability Assays.

Bacteria viability of BC and probiotic celluloses was qualitatively assessed by confocal laser scanning microscopy (CLSM). The samples were washed with sterile saline solution and stained with LIVE/DEAD BacLight Bacterial Viability Kit (ThermoFisher) following manufacturer's instructions. This assay combines membrane-impermeable DNA-binding stain, i.e. propidium iodide (PI), with membrane-permeable DNA-binding counterstain, SYTO9, to stain dead and live and dead bacteria, respectively. Cell viability along the BC matrix was evaluated with a confocal microscope (Nikon Eclipse Ti-E A1) equipped with 20× oil immersion objective. For acquiring SYTO 9 signals, 488 nm laser and 505-550 nm emission filter was used. For PI, 561 nm laser and 575 nm long-pass emission filter was used. Images were Analysed with NIS Elements Software.

Bacteria activity through pH monitoring and POM reduction capacity.

The metabolic activity of Lf and Lg on probiotic cellulose was evaluated by pH monitoring (HACH SensION™ pHmeter) and measuring its reductive capacity against electrochromic polyoxometalates (POM, [P₂Mo^{VI}₁₈O₆₂]⁶⁻), following a previously reported protocol (González A. et al., 2015, Chem. Commun. 51, 10119-10122). Briefly, probiotic cellulose samples were incubated in 100 mL of diluted MRS broth (1:10) in anaerobic conditions, at 37° C. and 180 rpm. At scheduled times (0, 1, 2, 4, 5, 7 and 20 h), 1 mL-aliquot was collected, centrifuged (3000 g, 5 min) and filtered with a 0.2 m filter to remove any residual bacteria. Then, 190 μL of the sample was mixed with 10 μL of POM solution (10 mM) on a 96-well and irradiated with UV light (365 nm) for 10 min. The absorbance at 820 nm was measured with a NanoQuant plate reader (Tecan). Data are expressed as mean of triplicates±standard deviations.

Inhibitory and Antimicrobial Activity of Probiotic Cellulose Against Staphylococcus aureus (SA) and Pseudomonas aeruginosa (PA).

Antimicrobial activity of non-encapsulated probiotics against Staphylococcus aureus (SA) and Pseudomonas aeruginosa (PA), two common pathogens involved in wound infection, was initially evaluated by an agar spot test in MRS (S. Tejero-Sarinena et al. 2012, Anaerobe 18, 530-538). In brief, overnight probiotic culture (10⁹ UFC mL⁻¹) were inoculated as a 5 μL spot on MRS agar plates (3 spots/plate). After 24 h of incubation at 37° C. under anaerobic conditions, the plates were overlaid with 6 mL of 0.7% (w/v) of tryptic soy agar (TSA) at 45° C., previously inoculated with 0.1 mL of an overnight culture of S. aureus or P. aeruginosa. The plates were incubated 24 h at 30° C. and 37° C., respectively, before examination of the corresponding inhibition zones.

After that, antimicrobial activity of probiotic cellulose and non-encapsulated probiotics was evaluated by an agar diffusion assay (Khalid A., et al. 2017, Carbohydr. Polym. 164, 214-221) and a modified time-kill test, both in pathogen-favourable tryptic soy media (Balouiri M. et al., 2016, J. Pharm. Anal. 6, 71-79). The agar diffusion assay was carried out as follow: 0.1 mL of an overnight culture of SA or PA was spread on TSA petri dishes. Then, Lf- and Lg-cellulose were placed on agar plates containing the selected bacterial strains and incubated 24 hours at pathogen optimal temperature (37° C. for PA, and 30° C. for SA) before examination of inhibition zones. In parallel, equivalent CFU of Lf and Lg was placed into the agar petri dish containing the pathogen, by using sterile cylinders. After 24 hours of incubation, the inhibition zones of non-encapsulated probiotics and probiotic cellulose were imaged and compared.

For the time-kill assays, Lg- and Lf-cellulose were introduced into tryptic soy broth (TSB) medium containing 7·10⁶ CFU of pathogen and incubated with orbital shaking for 24 h at 30° C. for SA and at 37° C. for PA (Wang Y. et al., 2017, Sci. Rep. 7, 1-9; Wang Y. et al., 2018, npj Biofilms Microboimes 4). The pathogen survival was assayed by the serial dilution method, plating on TSA in triplicate and 24 hours of incubation. The same protocol was followed for BC and for pathogen culture (controls).

### Example 1—Production of Probiotic Cellulose

Probiotic cellulose was produced through an innovative and smart strategy. It was based on the fact that, whereas the cellulose-producing bacterium Acetobacter xylinum (Ax) is strictly aerobic, the probiotics Lactobacillus fermentum (Lf) and Lactobacillus gasseri (Lg) are facultative anaerobic. The two selected probiotics, have activity related to the prevention and/or treatment of infections. Lf is an immune-stimulant that strengthens the microbiota, and Lg has exhibited antimicrobial activity against Staphylococcus aureus, one of the most common bacteria of chronic ulcers.

Gram stain of cellulose shed light on the growth mechanism of probiotic cellulose (FIG. 1A-D). Concretely, the co-culture of Gram-negative Ax and Gram-positive probiotic (Lg or Lf) in aerobic HS medium (optimum for Ax) resulted in the formation of a dense transparent cellulose film, containing both bacteria (Ax/Lf or Ax/Lg), being the facultative anaerobic probiotics located at the bottom (FIG. 1), as far as possible from the air-culture interface. The replacement of the media to MRS and the removal of oxygen (anaerobic conditions, optimal for probiotics) caused the proliferation of the probiotics, being the cellulose film fully invaded (FIG. 1C,D). Interestingly, the dose of L. fermentum can be tuned by the incubation time in anaerobic conditions. As expected, longer incubation times produce a greater number of L. fermentum (FIG. 1C-D).

BC produced aerobically in the presence of Ax and the probiotic is, in fact, a two-sided material. FESEM micrographs of the air-exposed face showed the typical fibrous morphology of Ax (FIG. 1E and FIG. 2A,B), whereas bacteria at the submerged face presented the typical bacilliform appearance of probiotics. In fact, FESEM micrographs of the cross-section (FIG. 1F) revealed two clearly differentiated areas: one exposed to air, containing exclusively Ax, and the other exposed to the bulk aqueous phase, which only included probiotics. When passed to a strictly anaerobic medium (optimal for probiotics), the probiotics extensively proliferated and invaded the entire cellulose matrix to such an extent that FESEM micrographs of both faces were similar (FIG. 1G and FIG. 2C,D). Under these latter conditions no evidences of reminiscent Ax were detected. Therefore, this material, probiotic cellulose, only contains probiotics, which are distributed throughout the cellulose network. Despite the high density of probiotics (FIG. 1G), i.e., 1.4·10¹¹ and 8.7·10¹⁰ CFU of Lf and Lg, respectively, per mg of cellulose, the entrapment did not affect the size of the cellulose nanofibres, which maintain diameters ranging between 20 and 90 nm (FIG. 3).

It is important to highlight that probiotic cellulose is produced by a one-pot synthesis, using mild conditions. In contrast, all previously reported bacterial cellulose and derivatives, first required the isolation of pure BC by a long and quite expensive procedure, based on successive treatments with ethanol and NaOH (or KOH) at high temperatures, to eliminate any rest of cellulose-producing bacteria. This is of paramount importance from an economical and environmental point of view for the industrial production. The synthetic process of probiotic cellulose is environmentally safe and fulfill with the principles of green chemistry, in contrast with the highly exploited conventional production of BC.

Similar results as those described in this example 1 were obtained when using B. breve instead of L. fermentum or L. gasseri (data not shown).

### Example 2—Viability of Entrapped Probiotics

Live/dead viability tests, based on the SYTO 9—propidium iodide fluorescent dyes, demonstrated the viability of the entrapped probiotics (FIG. 4). Confocal laser scanning microscopy (CLSM) image of the cellulose obtained after co-culture of Ax and Lf in aerobiosis contained a mixture of live bacteria with a high density of dead bacteria with fibrous (Ax) and short bacilliform (Lf) morphologies (FIG. 4A,B). Contrastingly, the probiotic cellulose showed an extremely high density of live probiotics, with very few dead bacteria (FIG. 4C,D). Moreover, the 3D CLSM image confirmed that probiotic cellulose is a homogeneous material, since live probiotics migrated and colonised the entire cellulose matrix after 48 h (FIG. 4D). Similar results were obtained with Lg-cellulose samples (data not shown).

### Example 3—Metabolic Activity of the Entrapped Probiotics

Evaluating the metabolic activity of the probiotics is highly relevant to assess the functionality of the biomaterial. Lf and Lg are lactic acid bacteria that excrete lactic acid (pka=3.86), which in turn determines the pH of the medium. In fact, the monitoring of the pH drop versus time is a genuine experiment to check the viability and activity of lactic acid bacteria (Tachedjian G. et al., 2017, Res. Microbiol. 168:782-792). As it is evident in FIG. 5A, when Lf- or Lg-cellulose were incubated in MRS medium, the pH continuously decreased until a pH value around 4, very close to the pkₐ of the lactic acid, which points out that probiotics are not only alive but also metabolically actives.

On the other hand, we reported that acid lactic bacteria act as electron donors to the electrochromic polyoxometalate [P₂Mo^{VI}₁₈O₆₂]⁶⁻ (POM) (Gonzalez A. et al., 2015, Chem. Commun. 51:10119-10122). We found that the reductive capacity of these lactic acid bacteria correlates to their metabolic activity, so that the more active the probiotic is, the more reduced form of POM develops. Aliquots of the Lf-cellulose or Lg-cellulose cultures media were mixed to water solutions of POM, and the temporal evolution of the intensity of the characteristic UV-vis absorption band at 820 nm of the reduced form of POM was monitored (FIG. 5B). POM reduction readily occurred and gradually increased with time, which is a complementary evidence of the metabolic activity of the probiotics once entrapped in probiotic cellulose materials.

Similar results as those described in this example 3 were obtained with cellulose comprising B. breve as probiotics instead of L. fermentum or L. gasseri (data not shown).

### Example 4—Antibacterial Activity

Both Lf and Lg present antimicrobial activity against SA and PA but in media favouring the growth and proliferation of probiotics, such as MRS (FIG. 6A). However, neither Lf nor Lg were able to inhibit the pathogenic growth in optimal pathogenic media such as tryptic soy agar (TSA) (FIG. 6B), which is indeed a more realistic scenario of infection.

With this in mind, we initially tested the antibacterial activity of probiotic cellulose using the disk diffusion set-up depicted in FIG. 7A, where the pathogens were dispersed in TSA. Even in these unfavourable conditions, probiotic celluloses produced inhibition zones against both pathogens (FIG. 7A).

These observations were confirmed by time-kill experiments. When SA or PA were cultivated in TSB (an unfavourable medium for probiotics), we found pathogens proliferation from initial loads of 10⁶-10⁷ to 10⁹ CFU after 24 h (FIG. 7B). Then, in a control experiment, we observed that addition of bacterial cellulose did not affect the pathogen proliferation (FIG. 8B, SA+BC or PA+BC bars). Nonetheless, when probiotic cellulose (either Lf- or Lg-cellulose) was added instead of bacterial cellulose, we witnessed a dramatic decline in pathogen viability. In particular, Lg-cellulose eliminated PA and SA after 24 hours, while Lf-cellulose practically killed PA and notably decreased SA viability.

### CONCLUSION

We have developed a new concept of antibiotic-free antibacterial—probiotic cellulose—which consists of bacterial cellulose loaded with live and active probiotics. The two probiotic celluloses (Lg- and Lf-cellulose) showed extraordinary antibacterial activity against Staphylococcus aureus and Pseudomonas aeruginosa, the two most active pathogens in severe skin infections. Furthermore, probiotic celluloses, in contrast to probiotics, exhibit antibacterial efficacy even in conditions that are favourable for pathogens and unfavourable for probiotics. Our smart strategy to produce probiotic cellulose can be extended to other facultative anaerobic probiotics and easily scaled for industrial production. In fact, the production of probiotic cellulose does not require the lengthy and quite expensive chemical treatments necessary to isolate bacterial cellulose. Probiotic cellulose is an antibiotic-free antibacterial agent with excellent practical application today, and tomorrow, in a hypothetical post-antibiotic era, where common infections and minor injuries could kill.

1. A biomaterial comprising a bacterial cellulose matrix and probiotics entrapped in said matrix, wherein the biomaterial is essentially free from bacteria that produce cellulose.

2. The biomaterial according to claim 1, wherein the bacterial cellulose has been produced by aerobic bacteria.

3. The biomaterial according to claim 2, wherein the aerobic bacteria are from the genus Acetobacter, Gluconacetobacter, Komagataeibacter or combinations thereof.

4. The biomaterial according to claim 3, wherein the aerobic bacteria from the genus Acetobacter are from the species A. xylinum, A. nitrogenifigens, A. orientalis or combinations thereof, the aerobic bacteria from the genus Gluconacetobacter are from the species G. hansenii, G. swingsii, G. sacchari, G. kombuchae, G. entanii, G. persimmonis, G. sucrofermentans or combinations thereof, the aerobic bacteria from the genus Komagataeibacter are from the species K. europaeus, K. medellinensis, K. intermedius, K. rhaeticus, K. kakiaceti, K. oboediens, K. nataicola, K. saccharivorans, K. maltaceti, or combinations thereof.

5. The biomaterial according to claim 4, wherein the aerobic bacteria from the genus Acetobacter are from the species Acetobacter xylinum, preferably from the strain deposited at the Colección Española de Cultivos Tipo (CECT) with accession number CECT 473.

6. The biomaterial according to any of claims 1-5, wherein the probiotics are facultative anaerobic bacteria and/or aerotolorant anaerobic bacteria.

7. The biomaterial according to claim 6, wherein probiotics are from the genus Lactobacillus, Bifidobacterium, Lactococcus, Streptococcus, or combinations thereof.

8. The biomaterial according to claim 7, wherein probiotics from the genus Lactobacillus are from the species L. fermentum, L. acidophilus, L. plantarum. L. rhamnosus, L. casei, L. johnsonii, L. delbrueckii, L. salivarus, or combinations thereof, probiotics from the genus Bifidobacterium are from the species B. breve, B. longum, B. animalis, B. infantum, B. animalis, or combinations thereof, probiotics from the genus Lactococcus are from the species L. lactis and probiotics from the genus Streptococcus are from the species S. thermophilus.

9. The biomaterial according to claim 8, wherein probiotics from the genus Lactobacillus are from the species Lactobacillus fermentum, Lactobacillus acidophilus, preferably from the strain CECT 903, Lactobacillus plantarum, preferably from the strain CECT 220, Lactobacillus rhamnosus, preferably from the strain CECT 278, or combinations thereof, and the probiotics from the genus Bifidobacterium are from the species Bifidobacterium breve.

10. A method for obtaining the biomaterial according to any of claims 1-9, comprising:
(i) culturing aerobic bacteria that produce cellulose simultaneously with facultative anaerobic probiotics and/or aerotolorant anaerobic probiotics under conditions suitable for the production of cellulose by the bacteria that produce cellulose, thereby resulting in a cellulose matrix containing the bacteria and the probiotics and,
(ii) incubating the cellulose matrix obtained in step (i) in a culture medium that provides conditions which are suitable for the proliferation of the probiotics in said matrix and which are not suitable the proliferation of the aerobic bacteria.

11. The method according to claim 10, wherein the aerobic bacteria are from the Genus Acetobacter, Gluconacetobacter Komagataeibacter, or combinations thereof.

12. The method according to claim 11, wherein the aerobic bacteria from the genus Acetobacter are from the species A. xylinum, A. nitrogenifigens, A. orientalis or combinations thereof, the aerobic bacteria from the genus Gluconacetobacter are from the species G. hansenii, G. swingsii, G. sacchari, G. kombuchae, G. entanii, G. persimmonis, G. sucrofermentans or combinations thereof, the aerobic bacteria from the genus Komagataeibacter are from the species K. europaeus, K. medellinensis, K. intermedius, K. rhaeticus, K. kakiaceti, K. oboediens, K. nataicola, K. saccharivorans, K. maltaceti or combinations thereof.

13. The method according to claim 12 wherein the aerobic bacteria from the genus Acetobacter are from the species Acetobacter xylinum, preferably from the strain deposited at the Colección Española de Cultivos Tipo (CECT) with accession number CECT 473.

14. The method according to any of claims 10-13 wherein probiotics are from the genus Lactobacillus, Bifidobacterium, Lactococcus, Streptococcus or combinations thereof.

15. The method according to claim 14, wherein probiotics from the genus Lactobacillus are from the species L. fermentum, L. acidophilus, L. plantarum, L. rhamnosus, L. casei, L. johnsonii, L. delbrueckii, L. salivarus or combinations thereof, probiotics from the genus Bifidobacterium are from the species B. breve, B. longum, B. animalis, B. infantum, B. animalis or combinations thereof, probiotics from the genus Lactococcus are from the species L. lactis and probiotics from the genus Streptococcus are from the species S. thermophiles.

16. The method according to claim 15, wherein probiotics from the genus Lactobacillus are from the species Lactobacillus fermentum, Lactobacillus acidophilus, preferably from the strain CECT 903, Lactobacillus plantarum, preferably from the strain CECT 220, Lactobacillus rhamnosus, preferably from the strain CECT 278, or combinations thereof, and probiotics from the genus Bifidobacterium are from the species Bifidobacterium breve.

17. The method according to any of claims 10-16 wherein step (i) is performed under aerobic conditions.

18. The method according to any of claims 10-17 wherein step (i) is performed in Hestrin-Schramm (HS) culture medium.1

19. The method according to any of claims 10-18 wherein the probiotics comprise bacteria from the genus Bifidobacterium, preferably from the species Bifidobacterium breve, and step (i) is performed in HS culture medium enriched with cysteine, preferably with about 5 μg/ml of cysteine.

20. The method according to any of claims 10-19 wherein step (i) is performed at 30° C.

21. The method according to any of claims 10-20 wherein step (i) is performed under static conditions or dynamic conditions.

22. The method according to any of claims 10-21 wherein step (i) is performed for at least one day.

23. The method according to any of claims 10-22 wherein an additional step of rising the cellulose is carried out between step (i) and (ii).

24. The method according to any of claims 10-23, wherein step (ii) is performed by incubating the cellulose obtained in step (i) in a culture medium under an anaerobic atmosphere.

25. The method according to claim 24, wherein the culture medium is MRS medium.

26. The method according to claim 25, wherein the probiotics comprise bacteria from the genus Bifidobacterium, preferably from the species Bifidobacterium breve and the culture media is MRS medium enriched in cysteine, preferably with about 5 μg/ml of cysteine.

27. The method according to any of claims 10-26 wherein step (ii) is performed at 37° C.

28. The method according to any of claims 10-27 wherein step (ii) is performed under static conditions or dynamic conditions.

29. The method according to any of claims 10-28 wherein step (ii) is performed for at least 1 day.

30. A biomaterial obtained by the method according to any of claims 10-29.

31. The biomaterial according to any of claims 1 to 9 or to claim 30 for use in medicine.

32. The biomaterial according to any of claims 1 to 9 or to claim 31, for use in the treatment of a wound or of a bacterial infection.

33. The biomaterial for use according to claim 32 wherein the infection is caused by Staphylococcus aureus or Pseudomonas aeruginosa.

34. The biomaterial for use according to any of claims 32 or 33, wherein the bacterial infection is selected from the group consisting of bacterial vaginosis, mastitis, and otitis, impetigo, ecthyma, erythema, erysipelas, bacterial cellulitis, folicullitis, furunculosis, hydrosadenitis, paronychia, infection in atopic dermatitis, superinfection in atopic dermatitis, ocular infection, infection of the urinary tract, infection of cardiac valves, infection of artificial cardiac valves, bone infection, joint infection, wound infection, and a burn infection.

35. A pharmaceutical composition comprising the biomaterial of any of claims 1-9 or 30, and a pharmaceutically acceptable carrier.

36. A coated food product which comprises:
(i) a biomaterial according to any of claims 1-9 or 30, and
(ii) an edible filling composition,
wherein the biomaterial (i) coats the filling composition (ii).

37. The coated food product according to claim 36, wherein the filling composition comprises animal matter, vegetables, cereals, fruits or combinations thereof.

38. The coated food product according to claim 37, wherein the filing composition comprises animal matter and said animal matter comprises red meat, pork, poultry, fish or combinations thereof.

39. A packaged medical device wherein the device is packaged in a container which comprises a biomaterial according to any of claims 1-9 or 30.

40. The packaged medical device of claim 39 wherein the medical device is a surgical device.

41. Use of the biomaterial according to any of claims 1-9 or 30 as a coat in a coated food product.

42. The use according to claim 41, wherein the coated food product comprises the edible filling composition as defined in claims 36-37.

43. Use of a biomaterial according to any of claims 1-9 or 30 for the packaging of a medical device.
